SEQUENCE LISTING (84 sequences)

SEQ ID NO: 1 - protein, 272 aa, Homo sapiens
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 2 - protein, 47 aa, Homo sapiens
KISHFLKMESLNFIRAHTPYINIYNCEPANPSEKNSPSTQYCYSIQS

SEQ ID NO: 3 - protein, 42 aa, Homo sapiens
LQMAGQCSQNEYFDSLLHACIPCQLRCSSNTPPLTCQRYCNA

SEQ ID NO: 4 - protein, 367 aa, Homo sapiens
FPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAG

SEQ ID NO: 5 - protein, 311 aa, Homo sapiens
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYA

SEQ ID NO: 6 - protein, 317 aa, Homo sapiens
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 7 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 8 - protein, 622 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 9 - protein, 133 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 133 aa, synthetic construct
APTSSSTKKTQLQLETLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 11 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 12 - protein, 240 aa, Homo sapiens
QEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSIL

SEQ ID NO: 13 - protein, 134 aa, Homo sapiens
LQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 14 - protein, 208 aa, Homo sapiens [sequence omitted]

SEQ ID NO: 15 - protein, 224 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 16 - protein, 50 aa, synthetic construct
  VARIANT 1..50: note = Wherein up to 9 copies of GSGGS may be deleted
GSGGSGSGGSGSGGSGSGGSGSGGSGSGGSGSGGSGSGGSGSGGSGSGGS

SEQ ID NO: 17 - protein, 50 aa, synthetic construct
  VARIANT 1..50: note = Wherein up to 9 copies of GGGGS may be deleted
GGGGSGGGGSGGGGSGGGGSGGGGSGGGGSGGGGSGGGGSGGGGSGGGGS

SEQ ID NO: 18 - protein, 4 aa, synthetic construct
GGSG

SEQ ID NO: 19 - protein, 5 aa, synthetic construct
GGSGG

SEQ ID NO: 20 - protein, 5 aa, synthetic construct
GSGSG

SEQ ID NO: 21 - protein, 5 aa, synthetic construct
GSGGG

SEQ ID NO: 22 - protein, 5 aa, synthetic construct
GGGSG

SEQ ID NO: 23 - protein, 5 aa, synthetic construct
GSSSG

SEQ ID NO: 24 - protein, 5 aa, synthetic construct
AAAGG

SEQ ID NO: 25 - protein, 100 aa, synthetic construct
  VARIANT 1..100: note = Wherein up to 19 copies of EAAAK may be deleted
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 26 - protein, 102 aa, synthetic construct
  VARIANT 2..101: note = Wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 27 - protein, 206 aa, synthetic construct
  VARIANT 2..101: note = Wherein up to 19 copies of EAAAK may be deleted
  VARIANT 106..205: note = Wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKALEAEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 28 - protein, 5 aa, synthetic construct
EAAAK

SEQ ID NO: 29 - protein, 10 aa, synthetic construct
EAAAKEAAAK

SEQ ID NO: 30 - protein, 15 aa, synthetic construct
EAAAKEAAAKEAAAK

SEQ ID NO: 31 - protein, 46 aa, synthetic construct
AEAAAKEAAAKEAAAKEAAAKALEAEAAAKEAAAKEAAAKEAAAKA

SEQ ID NO: 32 - protein, 12 aa, synthetic construct
AEAAAKEAAAKA

SEQ ID NO: 33 - protein, 5 aa, synthetic construct
PAPAP

SEQ ID NO: 34 - protein, 8 aa, synthetic construct
APAPAPAP

SEQ ID NO: 35 - protein, 12 aa, synthetic construct
APAPAPAPAPAP

SEQ ID NO: 36 - protein, 16 aa, synthetic construct
APAPAPAPAPAPAPAP

SEQ ID NO: 37 - protein, 20 aa, synthetic construct
APAPAPAPAPAPAPAPAPAP

SEQ ID NO: 38 - protein, 4 aa, synthetic construct
KPKP

SEQ ID NO: 39 - protein, 8 aa, synthetic construct
KPKPKPKP

SEQ ID NO: 40 - protein, 12 aa, synthetic construct
KPKPKPKPKPKP

SEQ ID NO: 41 - protein, 16 aa, synthetic construct
KPKPKPKPKPKPKPKP

SEQ ID NO: 42 - protein, 20 aa, synthetic construct
KPKPKPKPKPKPKPKPKPKP

SEQ ID NO: 43 - protein, 4 aa, synthetic construct
EPEP

SEQ ID NO: 44 - protein, 8 aa, synthetic construct
EPEPEPEP

SEQ ID NO: 45 - protein, 12 aa, synthetic construct
EPEPEPEPEPEP

SEQ ID NO: 46 - protein, 16 aa, synthetic construct
EPEPEPEPEPEPEPEP

SEQ ID NO: 47 - protein, 20 aa, synthetic construct
EPEPEPEPEPEPEPEPEPEP

SEQ ID NO: 48 - protein, 557 aa, Homo sapiens [sequence omitted]

SEQ ID NO: 49 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 50 - protein, 184 aa, Homo sapiens [sequence omitted]

SEQ ID NO: 51 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 52 - protein, 1255 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIVSAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERAKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 53 - protein, 580 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPS

SEQ ID NO: 54 - protein, 847 aa, Homo sapiens
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 55 - protein, 865 aa, Homo sapiens
MALVLGSLLLLGLCGNSFSGGQPSSTDAPKAWNYELPATNYETQDSHKAGPIGILFELVHIFLYVVQPRDFPEDTLRKFLQKAYESKIDYDKPETVILGLKIVYYEAGIILCCVLGLLFIILMPLVGYFFCMCRCCNKCGGEMHQRQKENGPFLRKCFAISLLVICIIISIGIFYGFVANHQVRTRIKRSRKLADSNFKDLRTLLNETPEQIKYILAQYNTTKDKAFTDLNSINSVLGGGILDRLRPNIIPVLDEIKSMATAIKETKEALENMNSTLKSLHQQSTQLSSSLTSVKTSLRSSLNDPLCLVHPSSETCNSIRLSLSQLNSNPELRQLPPVDAELDNVNNVLRTDLDGLVQQGYQSLNDIPDRVQRQTTTVVAGIKRVLNSIGSDIDNVTQRLPIQDILSAFSVYVNNTESYIHRNLPTLEEYDSYWWLGGLVICSLLTLIVIFYYLGLLCGVCGYDRHATPTTRGCVSNTGGVFLMVGVGLSFLFCWILMIIVVLTFVFGANVEKLICEPYTSKELFRVLDTPYLLNEDWEYYLSGKLFNKSKMKLTFEQVYSDCKKNRGTYGTLHLQNSFNISEHLNINEHTGSISSELESLKVNLNIFLLGAAGRKNLQDFAACGIDRMNYDSYLAQTGKSPAGVNLLSFAYDLEAKANSLPPGNLRNSLKRDAQTIKTIHQQRVLPIEQSLSTLYQSVKILQRTGNGLLERVTRILASLDFAQNFITNNTSSVIIEETKKYGRTIIGYFEHYLQWIEFSISEKVASCKPVATALDTAVDVFLCSYIIDPLNLFWFGIGKATVFLLPALIFAVKLAKYYRRMDSEDVYDDVETIPMKNMENGNNGYHKDHVYGIHNPVMTSPSQH

SEQ ID NO: 56 - protein, 1210 aa, Homo sapiens [sequence omitted]

SEQ ID NO: 57 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 58 - protein, 810 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 809 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 580 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSLQMAGQCSQNEYFDSLLHACIPCQLRCSSNTPPLTCQRYCNAAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 579 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSLQMAGQCSQNEYFDSLLHACIPCQLRCSSNTPPLTCQRYCNAAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 585 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSKISHFLKMESLNFIRAHTPYINIYNCEPANPSEKNSPSTQYCYSIQSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 584 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSKISHFLKMESLNFIRAHTPYINIYNCEPANPSEKNSPSTQYCYSIQSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 849 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 848 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 227 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 226 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 77 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 133 aa, Homo sapiens [sequence omitted]

SEQ ID NO: 80 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 81 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 82 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 83 - protein, 40 aa, synthetic construct
  VARIANT 1..40: note = Wherein up to 9 copies of GGGS may be deleted
GGGSGGGSGGGSGGGSGGGSGGGSGGGSGGGSGGGSGGGS

SEQ ID NO: 84 - protein, 5 aa, synthetic construct
  VARIANT 4: note = X is any amino acid except Proline
VPGXG

(19) United States
(12) Patent Application Publication
(10) Pub. No.: US 2025/0161355 A1
(43) Pub. Date: May 22, 2025

(54) PROTEINS FOR MODULATING CELL THERAPY PRODUCTS AND METHODS OF USE THEREOF

(71) Applicant: Cue Biopharma, Inc., Boston, MA (US)

(72) Inventor: Anish Suri, Boston, MA (US)

(21) Appl. No.: 18/193,443

(22) Filed: Mar. 30, 2023

Related U.S. Application Data
(60) Provisional application No. 63/327,715, filed on Apr. 5, 2022.

(51) Int. Cl. A61K 35/17; A61P 35/00; C07K 14/47; C07K 14/55; C07K 14/715; C12N 15/85

(52) U.S. Cl. CPC A61K 35/17; A61P 35/00; C07K 14/4702; C07K 14/55; C07K 14/7155; C12N 15/85

(57) ABSTRACT

This disclosure provides modulatory polypeptides (“CT-MP”) that bind to and modulate cell therapy products such as CAR-T cells that comprise antigen binding polypeptides, e.g., CARs or other polypeptides that bind cancer-associated antigens. This disclosure further provides methods of increasing the number and/or activity of such cell therapy products, as well as methods of treating cancer.

### CROSS-REFERENCE

This application claims the benefit of U.S. Provisional Patent Application No. 63/327,715, filed Apr. 5, 2022, which application is incorporated herein by reference in its entirety.

### INCORPORATION-BY-REFERENCE OF MATERIAL ELECTRONICALLY SUBMITTED

A Sequence Listing is provided herewith as a Sequence Listing XML, “CUEB-149_SEQ_LIST_JUNE_2023” created on Jun. 2, 2023 and having a size of 97,606 bytes. The contents of the Sequence Listing XML are incorporated by reference herein in their entirety.

### INTRODUCTION

Cell therapy products include T cells that are modified to express proteins that bind to proteins on cancer cells. For example, “CAR-T cells” comprise a chimeric antigen receptor (“CAR”) that targets tumor-associated antigens. CAR-T cells can kill tumor cells and have been used successfully to treat cancers. For example, CAR-T cells targeting CD19, BCMA, CD30, CD22, or CD20 have shown significant activity in clinical studies. CAR-T therapies can suffer from drawbacks, however. Due to the large number of CAR-T cells that are administered, patients typically require a chemotherapy regimen for lymphodepletion prior to administration of the CAR-T cells. Also, to maintain therapeutic levels of CAR-T cells following administration, patients also may require treatment with aldesleukin (Proleukin®), which can result in serious adverse side effects. Further, some patients may not achieve their desired or optimal result due to suboptimal expansion or persistence of CAR-T cells in vivo.

### SUMMARY

This disclosure provides proteins for modulating cell therapy products such as CAR-T cells that comprise antigen binding polypeptides, e.g., CARs or other polypeptides that bind cancer-associated antigens. Cell therapy modulatory proteins of this disclosure are referred to herein as “CT-MP” (singular) or “CT-MPs” (plural). This disclosure further provides methods of increasing the number and/or activity of cell therapy products such as CAR-T cells, as well as methods of treating cancer.

### BRIEF DESCRIPTION OF THE DRAWINGS

FIGS. 1A-1F provide schematic depictions of examples of CT-MPs.

FIGS. 2A-2B provide amino acid sequences of CD19 (SEQ ID NOs: 48 and 1, respectively).

FIG. 3 provides an amino acid sequence of CD20.

FIG. 4 provides an amino acid sequence of BCMA.

FIG. 5 provides an amino acid sequence of CD30.

FIGS. 6A-6B provide amino acid sequences of Her2 (SEQ ID NOs: 52 and 53, respectively).

FIG. 7 provides an amino acid sequence of CD22.

FIG. 8 provides an amino acid sequence of CD133.

FIG. 9 provides an amino acid sequence of EGFR.

FIG. 10 provides an amino acid sequence of AFP.

FIGS. 11A-11B provide amino acid sequences of exemplary CT-MPs (SEQ ID NOs: 58 and 59, respectively).

FIGS. 12A-12B provide amino acid sequences of exemplary CT-MPs (SEQ ID NOs: 60 and 61, respectively).

FIGS. 13A-13B provide amino acid sequences of exemplary CT-MPs (SEQ ID NOs: 62 and 63, respectively).

FIGS. 14A-14B provide amino acid sequences of exemplary CT-MPs (SEQ ID NOs: 58 S6 64 and 65, respectively).

FIGS. 15A-15M provide amino acid sequences of exemplary immunoglobulin (Ig) Fc polypeptides (SEQ ID NOs: 66-78, respectively).

FIGS. 16A-16D provide amino acid sequences of a wild-type IL-2 polypeptide (FIG. 16A, SEQ ID NO:79), IL-2Rα (FIG. 16B, SEQ ID NO:80), IL-2Rβ (FIG. 16C, SEQ ID NO:81), and IL-2Rγ (FIG. 16D, SEQ ID NO:82).

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined BLAST, which is available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. Unless otherwise stated, “sequence identity” as referred to herein is determined by BLAST (Basic Local Alignment Search Tool) with the default settings selected.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The term “T cell modulatory polypeptide” (also referred to as a “TMP”), as used herein, includes a polypeptide on an antigen presenting cell (APC) (e.g., a dendritic cell, a B cell, and the like) that specifically binds a cognate polypeptide on a T cell, thereby providing a signal that mediates a T cell response, including, but not limited to, proliferation, activation, differentiation, and the like. A TMP can include, but is not limited to, IL-2, 4-1BBL, CD80, CD86, OX40L, inducible costimulatory ligand (ICOS-L), and the like.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. Also encompassed by the term are Fab′, Fv, F(ab′)₂, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “humanized immunoglobulin” as used herein refers to an immunoglobulin comprising portions of immunoglobulins of different origin, wherein at least one portion comprises amino acid sequences of human origin. For example, the humanized antibody can comprise portions derived from an immunoglobulin of nonhuman origin with the requisite specificity, such as a mouse, and from immunoglobulin sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized immunoglobulin is an immunoglobulin containing one or more immunoglobulin chains comprising a complementarity-determining region (CDR) derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., U.S. Pat. No. 4,816,567; European Patent No. 0,125,023 B1; U.S. Pat. No. 4,816,397; European Patent No. 0,120,694 B1; WO 86/01533; European Patent No. 0,194,276 B1; U.S. Pat. No. 5,225,539; European Patent No. 0,239,400 B1; and European Patent Application No. 0,519,596 A1. See also, U.S. Pat. Nos. 4,946,778; 5,476,786; and Bird et al. (1988) Science 242:423, regarding single chain antibodies.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (1996) Nature Structural Biol. 3:803; and Desmyter et al. (2015) Curr. Opin. Struct. Biol. 32:1). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

Unless indicated otherwise, the term “substantially” is intended to encompass both “wholly” and “largely but not wholly”. For example, an Ig Fc that “substantially does not induce ADCC” means an Ig Fc that induces no ADCC at all or that largely does not induce ADCC.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

Before the present disclosure is further described, it is to be understood that this disclosure is not limited to particular embodiments described, as such embodiments may, of course, vary. It is also to be understood that the terminology used herein is for the purpose of describing particular embodiments only, and is not intended to be limiting, since the scope of this disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of this disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a T-cell modulatory polypeptide” or “TMP” includes a plurality of such T-cell modulatory polypeptides and reference to “the cancer-associated antigen” or “CAA” includes reference to one or more cancer-associated antigens and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by this disclosure and are disclosed herein just as if each and every combination was individually and explicitly disclosed. In addition, all sub-combinations of the various embodiments and elements thereof are also specifically embraced by this disclosure and are disclosed herein just as if each and every such sub-combination was individually and explicitly disclosed herein.

The publications discussed herein are provided solely for their disclosure prior to the filing date of the present application. Nothing herein is to be construed as an admission that this disclosure is not entitled to antedate such publication by virtue of prior invention. Further, the dates of publication provided may be different from the actual publication dates which may need to be independently confirmed.

### DETAILED DESCRIPTION

This disclosure provides CT-MPs that bind to and modulate cell therapy products such as CAR-T cells that comprise antigen binding polypeptides, e.g., CARs or other polypeptides that bind cancer-associated antigens. This disclosure further provides methods of increasing the number and/or activity of cell therapy products such as CAR-T cells, as well as methods of treating cancer.

### CAR-T Cell Modulatory Proteins

A CT-MP as described herein binds to and modulates the activity of cell therapy products such as CAR-T cells that comprise antigen-binding polypeptides, e.g., CARs or other polypeptides that bind cancer-associated antigens. Although this disclosure is primarily directed to CT-MPs that bind to and modulate CAR-T cells, it will be readily understood that the CT-MPs described herein also are capable of binding to and modulating the activity of other T cells that have been modified to display antigen-binding polypeptides.

It is well known in the art that a CAR-T cell is a T cell that has been genetically modified to produce a CAR that is displayed on the surface of the CAR-T cell. A CAR typically consists of (i) an extracellular recognition domain, such as a single-chain Fv (scFv) or a nanobody, that binds to a cancer-associated antigen (CAA) on a cancer cell; (ii) structural components such as hinge and transmembrane domains; (iii) a costimulatory domain (such as CD28, 4-1BB, or OX40) that induce cytokine production and T cell proliferation; and (iv) an intracellular signaling domain (e.g., CD3-zeta (CD3ζ) or 4-1BB zeta) that activates T cells upon antigen binding. A CAR-T cell recognizes a CAA on the surface of a cancer cell, and provides anti-cancer cell effects such as direct cell killing (cytotoxic effects) and production of secreted factors (e.g., cytokines, interleukins, growth factors) that promote anti-cancer cell activity by other (resident) immune cells.

A CT-MP of this disclosure comprises: a) at least one T cell modulating polypeptide (“TMP”); b) at least one polypeptide that binds to an antigen binding polypeptide displayed by a T cell, for example, a CAR-binding polypeptide (“CAR-BP”) that specifically binds to an antigen-binding portion of a target CAR present on a CAR-T cell; and c) a scaffold component, e.g., an immunoglobulin (Ig) Fc polypeptide. The TMP is targeted to a CAR-T cell by virtue of the CAR-BP on the CT-MP. The TMP can modulate the activity of the CAR-T cell, e.g., causing activation and/or proliferation of a target CAR-T cell in vitro or in vivo. Where a TMP causes a target CAR-T cell to proliferate, for example, administration of the CT-MP in vivo before, concurrently with, and/or following administration of the CAR-T cells may make it possible to substantially reduce the number of CAR-T cells that are administered to an individual for anti-cancer treatment, thereby potentially reducing or eliminating the need for lymphodepletion therapy prior to administration of the CAR-T cells. Likewise, administration of the CT-MP in vivo following administration of the CAR-T cells may make it possible to substantially reduce or eliminate the need for IL-2 therapy following administration of the CAR-T cells. Additionally, CT-MPs may be used in the process of preparing a quantity of CAR-T cells, e.g., by helping to proliferate the CAR-T cells in vitro.

For example, a CT-MP can be administered to an individual at the same time as the CAR-T cells, either by administration of combination of the CAR-T cells and the CT-MP, or by sequential administration, or may administered at different times, e.g., the CT-MP is administered at one or more intervals following administration of the CAR-T cells. In such situations, the CT-MP effects an increase in the number of CAR-T cells in the individual in vivo.

Currently, CAR-T cell therapy may involve lymphodepleting chemotherapy before administration of CAR-T cells. Such lymphodepleting chemotherapy is carried out to decrease the number of resident T cells, so as to accommodate the large number of CAR-T cells that are administered. Because a CT-MP can induce proliferation of CAR-T cells in vivo, the number of CAR-T cells that are administered can be decreased, thus reducing or substantially eliminating the need for lymphodepleting chemotherapy. Furthermore, the TMP of a CT-MP can activate a CAR-T cell, which may in turn increase the anti-cancer activity of the CAR-T cell. Activation of a CAR-T cell thus may provide, e.g., one or both of (i) increased direct killing of a cancer cell, and (ii) increased secretion of cytokines that promote anti-cancer activity of resident immune cells.

As noted above, in some CAR-T cell therapy protocols, a cytokine such as recombinantly produced IL-2, e.g., aldesleukin (Proleukin®) is administered to an individual after administration of CAR-T cells in order to proliferate and/or activate CAR-T cells in vivo. Such IL-2 is administered systemically and can produce well-known and potentially serious adverse side effects. By providing a TMP to a target CAR-T cell, a CT-MP can reduce or substantially eliminate the need for administration of drugs such as recombinantly produced IL-2. In addition, because the TMP of a CT-MP is targeted to a target CAR-T cell to which the CT-MP binds, adverse side effects associated with systemic administration of the TMP (e.g., wild-type or variant IL-2) are reduced or substantially eliminated.

In some cases, a CT-MP, when contacted in vitro or in vivo with a target CAR-T cell, increases proliferation of the target CAR-T cell. For example, in some cases, a CT-MP, when contacted in vitro or in vivo with a target CAR-T cell, increases proliferation of the target CAR-T cell by at least 10%, at least 15%, at least 20%, at least 25%, at least 30%, at least 35%, at least 40%, at least 45%, at least 50%, at least 75%, at least 100% (or 2-fold), at least 2.5-fold, at least 5-fold, at least 10-fold, at least 50-fold, at least 100-fold, or more than 100-fold, compared to the proliferation of the target CAR-T cell not contacted with the CT-MP. In some cases, following contact with a CT-MP, the number of target CAR-T cells increases by at least 10%, at least 15%, at least 20%, at least 25%, at least 30%, at least 35%, at least 40%, at least 45%, at least 50%, at least 75%, at least 100% (or 2-fold), at least 2.5-fold, at least 5-fold, at least 10-fold, at least 50-fold, at least 100-fold, or more than 100-fold, compared to the number of target CAR-T cells in the absence of contact with the CT-MP.

In some cases, a CT-MP, when contacted with a target CAR-T cell, activates the target CAR-T cell.

A CT-MP does not include major histocompatibility complex (MHC) polypeptides (e.g., MHC class I polypeptides; MHC class II polypeptides; non-classical MHC polypeptides). For example, a CT-MP does not include MHC class I polypeptides such as an MHC class I heavy chain polypeptide or a 02-microglobulin (02M) polypeptide. A CT-MP is not designed to engage the T cell receptor (TCR) of a T cell. Rather, the CT-MP is designed to bind, via the CAR-BP, to the CAR of a CAR-T cell.

### CAR-Binding Polypeptides (CAR-BPs)

As noted above, a CT-MP comprises one or more CAR-BPs. Suitable CAR-BPs include antibodies that specifically bind to a CAR. Where a CAR-BP is an antibody, such an antibody can be an antigen-binding fragment of an antibody. For example, suitable antibodies include a single-chain Fv, a nanobody, and the like. Suitable CAR-BPs also include cancer-associated antigens to which the CAR binds.

For example, CAR-T therapies include Yescarta® (axicabtagene ciloleucel) is a CAR comprising a scFv that binds CD19. As further examples, Tecartus® (brexucabtagene autoleucel) is a CAR comprising a scFv that binds CD19; Kymriah® (tisagenleucleucel) is a CAR comprising a scFv that binds CD19; and Abecma® (idecabtagene vicleucel) comprising a scFv that binds BCMA; and Breyanzi® (lisocabtagene maraleucel) is a CAR comprising a scFv that binds CD19. Accordingly, a CAR-BP for one of these could comprise an antibody to the CAR or a CAA to which the CAR binds.

### Cancer-Associated Antigens

In some cases, a CAR-BP suitable for inclusion in a CT-MP is a cancer-associated antigen (CAA). The CAA can present one or more epitopes that are recognized by a CAR.

CAAs that can be included in a CT-MP include, but are not limited to, NY-ESO (New York Esophageal Squamous Cell Carcinoma 1), MART-1 (melanoma antigen recognized by T cells 1, also known as Melan-A), HPV (human papilloma virus) E6, BCMA (B-cell maturation antigen), CD123, CD133, CD171, CD19, CD20, CD22, CD30, CD33, CEA (carcinoembryonic antigen), EGFR (epidermal growth factor receptor), EGFRvIII (epidermal growth factor receptor variant III), EpCAM (epithelial cell adhesion molecule), EphA2 (ephrin type-A receptor 2), disialoganglioside GD2, GPC3 (glypican-3), HER2, IL13Ralpha2 (Interleukin 13 receptor subunit alpha-2), LeY (a difucosylated type 2 blood group-related antigen), MAGE-A1 (melanoma-associated antigen A1), MAGE-A3 (melanoma-associated antigen A3), MAGE-A4 (melanoma-associated antigen A4), melanoma glycoprotein, mesothelin, MUC1 (mucin 1), MUC16 (mucin-16), myelin, NKG2D (Natural Killer Group 2D) ligands, PSMA (prostate specific membrane antigen), and ROR1 (type I receptor tyrosine kinase-like orphan receptor).

CAAs that can be included in a CT-MP include, but are not limited to, 17-1A-antigen, alpha-fetoprotein (AFP), alpha-actinin-4, A3, antigen specific for A33 antibody, ART-4, B7, Ba 733, BAGE, bcl-2, bcl-6, BCMA, BrE3-antigen, CA125, CAMEL, CAP-1, carbonic anhydrase IX (CAIX), CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CD171, CDC27, CDK-4/m, CDKN2A, CEA, CEACAM5, CEACAM6, claudin (e.g., claudin-1, claudin-10, claudin-18 (e.g., claudin-18, isoform 2)), complement factors (such as C3, C3a, C3b, C5a and C5), colon-specific antigen-p (CSAp), c-Met, CTLA-4, CXCR4, CXCR7, CXCL12, DAM, Dickkopf-related protein (DKK), ED-B fibronectin, epidermal growth factor receptor (EGFR), EGFRvIII, EGP-1 (TROP-2), EGP-2, ELF2-M, Ep-CAM, EphA2, EphA3, fibroblast activation protein (FAP), fibroblast growth factor (FGF), Flt-1, Flt-3, folate binding protein, folate receptor, G250 antigen, gangliosides (such as GC2, GD3 and GM2), GAGE, GD2, gp100, GPC3, GRO-13, HLA-DR, HM1.24, human chorionic gonadotropin (HCG) and its subunits, HER2, HER3, HMGB-1, hypoxia inducible factor (HIF-1), HIF-1a, HSP70-2M, HST-2, Ia, IFN-gamma, IFN-alpha, IFN-beta, IFN-X, IL-4R, IL-6R, IL-13R, IL13Ralpha2, IL-15R, IL-17R, IL-18R, IL-2, IL-6, IL-8, IL-12, IL-15, IL-17, IL-18, IL-23, IL-25, ILGF, ILGF-1R, insulin-like growth factor-1 (IGF-1), IGF-1R, integrin αVβ3, integrin α5β1, KC4-antigen, killer-cell immunoglobulin-like receptor (KIR), KRAS, KS-1-antigen, KS1-4, LDR/FUT, Le^{gamma} macrophage migration inhibitory factor (MIF), MAGE, MAGE-3, MART-1, MART-2, mCRP, MCP-1, melanoma glycoprotein, mesothelin, MIP-IA, MIP-1B, MIF, mucins (such as MUC1, MUC2, MUC3, MUC4, MUC5ac, MUC13, MUC16, MUM-1/2 and MUM-3), NCA66, NCA95, NCA90, Nectin-4, NY-ESO-1, PAM4 antigen, pancreatic cancer mucin, PD-1, PD-L1, PD-1 receptor, placental growth factor, p53, PLAGL2, prostatic acid phosphatase, PSA, PRAME, PSMA, P1GF, RSS, RANTES, SAGE, 5100, survivin, survivin-2B, T101, TAC, TAG-72, tenascin, Thomson-Friedenreich antigens, Tn antigen, TNF-alpha, tumor necrosis antigens, TRAG-3, TRAIL receptors, vascular endothelial growth factor (VEGF), VEGF receptor (VEGFR) and WT-1.

In some cases, the CAA is an antigen associated with a hematological cancer. Examples of such antigens include, but are not limited to, BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, IGF-R1, IL6, KIR, PD-1, PD-L1, TRAILR1, TRAILR2, transferrin receptor, and VEGF. In some cases, the CAA is an antigen expressed by malignant B cells, such as CD19, CD20, CD22, CD25, CD38, CD40, CD45, CD74, CD80, CTLA-4, IGF-R1, IL6, PD-1, TRAILR2, or VEGF.

In some cases, the CAA is an antigen associated with a solid tumor. Examples of such antigens include, but are not limited to, CAIX, cadherins, CEA, c-MET, CTLA-4, EGFR family members, EpCAM, EphA3, FAP, folate-binding protein, FR-alpha, gangliosides (such as GC2, GD3 and GM2), HER2, HER3, IGF-1R, integrin αVβ3, integrin α5β1, Le^{gamma}, Liv1, mesothelin, mucins, NaPi2b, PD-1, PD-L1, PD-1 receptor, pgA33, PSMA, RANKL, ROR1, TAG-72, tenascin, TRAILR1, TRAILR2, VEGF, VEGFR, and others listed above. In some cases, the CAA is a solid tumor-associated antigen selected from: EGFR, HER2, EGFR806, mesothelin, PSCA, MUC1, claudin 18.2, EpCAM, GD2, VEGFR2, AFP, Nectin4/FAP, CEA, LewisY, Glypican-3, EGFRIII, IL-13Rα2, CD171, MUC16, PSMA, AXL, CD20, CD80/86, c-MET, DLL-3, DR5, EpHA2, FR-α, gp100, MAGE-A1, MAGE-A3, MAGE-A4, NY-ESO-1 and LMP1. See, e.g., Marofi et al. (2021) Stem Cell Res. Ther. 12:81.

In some cases, a CAA that is included in a CT-MP is selected from the group consisting of CD19, CD22, CD30, CD33, CD138, CD171, CEA, epidermal growth factor receptor, EFGRvIII, ErbB, FAP, GD2, Glypican 3, Her2, mesothelin, and NKG2D.

In some cases, a CAA that is included in a CT-MP comprises only the extracellular portion (e.g., an ectodomain) of a CAA.

### CD19 CAR-BPs

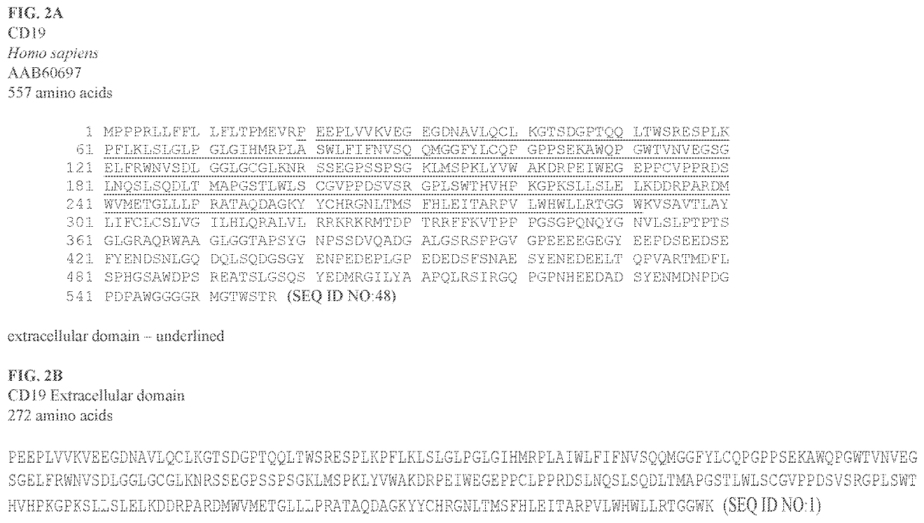

As one example, a CAA present in a CT-MP can comprise an amino acid sequence having at least 80%, at least 85%, at least 90%, at least 95%, at least 98%, at least 99%, or 100%, amino acid sequence identity to a contiguous stretch of from 25 amino acids to 557 amino acids (e.g., from 25 amino acids to 50 amino acids, from 50 amino acids to 100 amino acids, from 100 amino acids to 150 amino acids, from 150 amino acids to 200 amino acids, from 200 amino acids to 250 amino acids, from 250 amino acids to 300 amino acids, from 300 amino acids to 350 amino acids, from 350 amino acids to 400 amino acids, from 400 amino acids to 450 amino acids, from amino acids to 500 amino acids, or from 500 amino acids to 557 amino acids) of the CD19 amino acid sequence depicted in FIG. 2A. As another example, a CAA present in a CT-MP comprises an amino acid sequence having at least 80%, at least 85%, at least 90%, at least 95%, at least 98%, at least 99%, or 100%, amino acid sequence identity to a contiguous stretch of from 25 amino acids to 272 amino acids (e.g., from 25 amino acids to 50 amino acids, from 50 amino acids to 100 amino acids, from 100 amino acids to 150 amino acids, from 150 amino acids to 200 amino acids, from 200 amino acids to 250 amino acids, or from 250 amino acids to 272 amino acids) of an extracellular domain of CD19, e.g., of the following amino acid sequence:

(SEQ ID NO: 1: also depicted in FIG. 2B)
PEEPLVVKVEEGDNAVLQCLKGTSDGPTQQLTWSRESPLKPFLKLSLGL
PGLGIHMRPLAIWLFIFNVSQQMGGFYLCQPGPPSEKAWQPGWTVNVEG
SGELFRWNVSDLGGLGCGLKNRSSEGPSSPSGKLMSPKLYVWAKDRPEI
WEGEPPCLPPRDSLNQSLSQDLTMAPGSTLWLSCGVPPDSVSRGPLSWT
HVHPKGPKSLLSLELKDDRPARDMWVMETGLLLPRATAQDAGKYYCHRG
NLTMSFHLEITARPVLWHWLLRTGGWK.

### CD20 CAR-BPs

As another example, a CAA present in a CT-MP can comprise an amino acid sequence having at least 80%, at least 85%, at least 90%, at least 95%, at least 98%, at least 99%, or 100%, amino acid sequence identity to a contiguous stretch of from 25 amino acids to 297 amino acids (e.g., from 25 amino acids to 50 amino acids, from 50 amino acids to 100 amino acids, from 100 amino acids to 150 amino acids, from 150 amino acids to 200 amino acids, from 200 amino acids to 250 amino acids, or from 250 amino acids to 297 amino acids) of the CD20 amino acid sequence depicted in FIG. 3. As another example, a CAA present in a CT-MP comprises an amino acid sequence having at least 80%, at least 85%, at least 90%, at least 95%, at least 98%, at least 99%, or 100%, amino acid sequence identity to a contiguous stretch of from 25 amino acids to 47 amino acids of the following amino acid sequence:

(SEQ ID NO: 2)
KISHFLKMESLNFIRAHTPYINIYNCEPANPSEKNSPSTQYCYSIQS.

### BCMA CAR-BPs

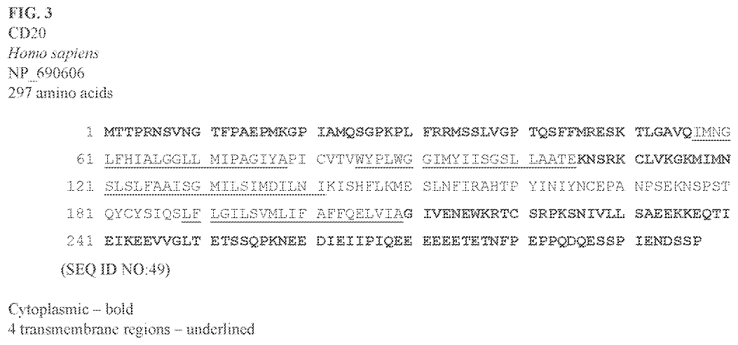

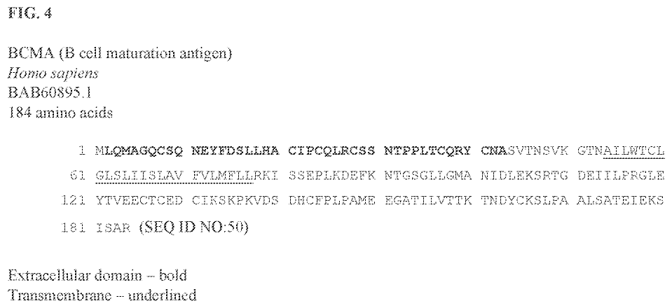

As another example, a CAA present in a CT-MP can comprise an amino acid sequence having at least 80%, at least 85%, at least 90%, at least 95%, at least 98%, at least 99%, or 100%, amino acid sequence identity to a contiguous stretch of from 25 amino acids to 184 amino acids (e.g., from 25 amino acids to 50 amino acids, from 50 amino acids to 100 amino acids, from 100 amino acids to 150 amino acids, or from 150 amino acids to 184 amino acids) of the BCMA amino acid sequence depicted in FIG. 4. As another example, a CAA present in a CT-MP comprises an amino acid sequence having at least 80%, at least 85%, at least 90%, at least 95%, at least 98%, at least 99%, or 100%, amino acid sequence identity to the following amino acid sequence: LQMAGQCSQNEYFDSLLHACIPCQLRCSSNTPPLTCQRYCNA (SEQ ID NO:3) and has a length of from 25 amino acids to 42 amino acids.

### CD30 CAR-BPs

As another example, a CAA present in a CT-MP can comprise an amino acid sequence having at least 80%, at least 85%, at least 90%, at least 95%, at least 98%, at least 99%, or 100%, amino acid sequence identity to a contiguous stretch of from 25 amino acids 595 amino acids (e.g., from 25 amino acids to 50 amino acids, from 50 amino acids to 100 amino acids, from 100 amino acids to 150 amino acids, from 150 amino acids to 200 amino acids, from 200 amino acids to 250 amino acids, from 250 amino acids to 300 amino acids, from 300 amino acids to 350 amino acids, from 350 amino acids to 400 amino acids, from 400 amino acids to 450 amino acids, from amino acids to 500 amino acids, from 500 amino acids to 550 amino acids, or from 550 amino acids to 595 amino acids) of the CD30 amino acid sequence depicted in FIG. 5. As another example, a CAA present in a CT-MP comprises an amino acid sequence having at least 80%, at least 85%, at least 90%, at least 95%, at least 98%, at least 99%, or 100%, amino acid sequence identity to a contiguous stretch of from 25 amino acids to 367 amino acids (e.g., from 25 amino acids to 50 amino acids, from 50 amino acids to 100 amino acids, from 100 amino acids to 150 amino acids, from 150 amino acids to 200 amino acids, from 200 amino acids to 250 amino acids, from 250 amino acids to 300 amino acids, from 300 amino acids to 350 amino acids, or from 350 amino acids to 367 amino acids) of the following amino acid sequence:

(SEQ ID NO: 4)
FPQDRPFEDTCHGNPSHYYDKAVRRCCYRCPMGLFPTQQCPQRPTDCRK
QCEPDYYLDEADRCTACVTCSRDDLVEKTPCAWNSSRVCECRPGMFCST
SAVNSCARCFFHSVCPAGMIVKFPGTAQKNTVCEPASPGVSPACASPEN
CKEPSSGTIPQAKPTPVSPATSSASTMPVRGGTRLAQEAASKLTRAPDS
PSSVGRPSSDPGLSPTQPCPEGSGDCRKQCEPDYYLDEAGRCTACVSCS
RDDLVEKTPCAWNSSRTCECRPGMICATSATNSCARCVPYPICAAETVT
KPQDMAEKDTTFEAPPLGTQPDCNPTPENGEAPASTSPTQSLLVDSQAS
KTLPIPTSAPVALSSTGKPVLDAG.

### Her2 (ERBB2) CAR-BPs

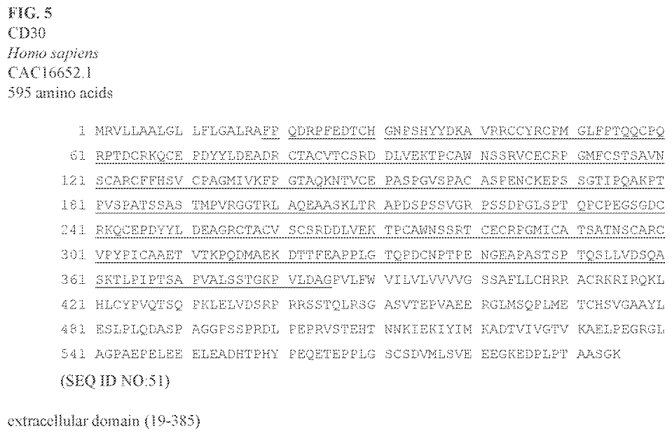

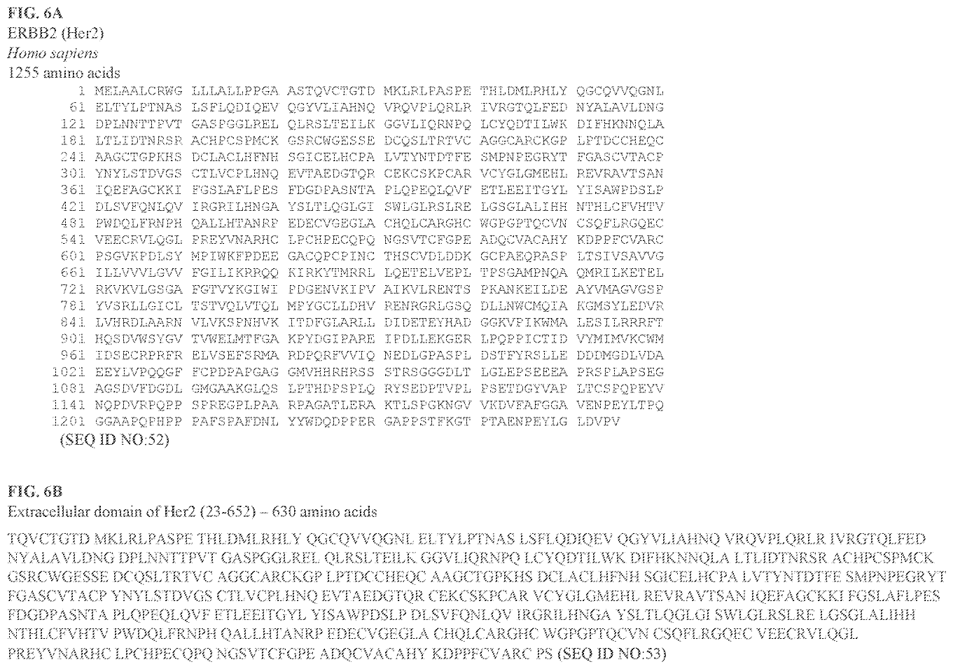

As another example, a CAA present in a CT-MP can comprise an amino acid sequence having at least 80%, at least 85%, at least 90%, at least 95%, at least 98%, at least 99%, or 100%, amino acid sequence identity to a contiguous stretch of from 25 amino acids to 652 amino acids (e.g., from 25 amino acids to 50 amino acids, from 50 amino acids to 100 amino acids, from 100 amino acids to 150 amino acids, from 150 amino acids to 200 amino acids, from 200 amino acids to 250 amino acids, from 250 amino acids to 300 amino acids, from 300 amino acids to 350 amino acids, from 350 amino acids to 400 amino acids, from 400 amino acids to 450 amino acids, from amino acids to 500 amino acids, from 500 amino acids to 550 amino acids, from 550 amino acids to 600 amino acids, or from 600 amino acids to 630 amino acids) of the Her2 extracellular domain amino acid sequence depicted in FIG. 6B.

### CD22 CAR-BPs

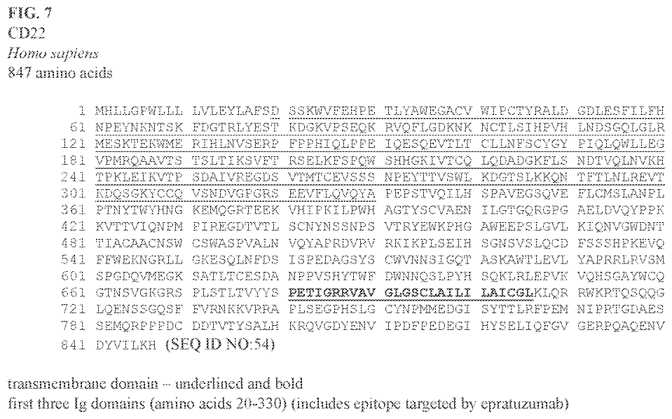

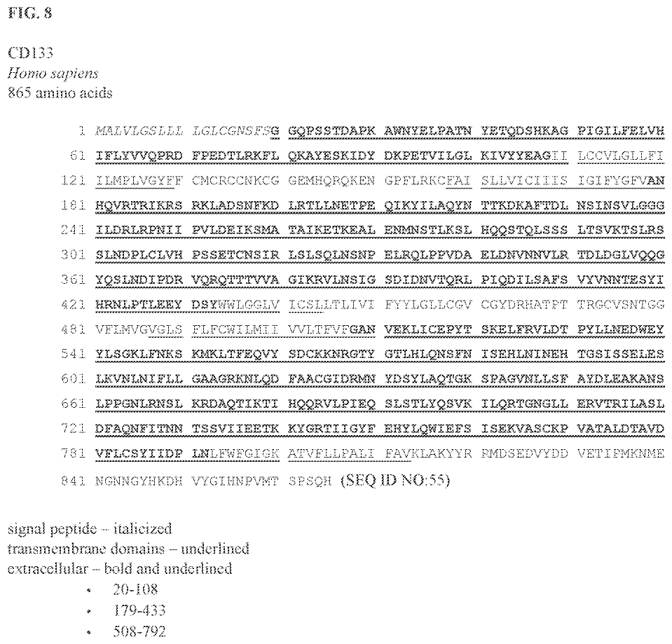

As another example, a CAA present in a CT-MP can comprise an amino acid sequence having at least 80%, at least 85%, at least 90%, at least 95%, at least 98%, at least 99%, or 100%, amino acid sequence identity to a contiguous stretch of from 25 amino acids to 847 amino acids (e.g., from 25 amino acids to 50 amino acids, from 50 amino acids to 100 amino acids, from 100 amino acids to 150 amino acids, from 150 amino acids to 200 amino acids, from 200 amino acids to 250 amino acids, from 250 amino acids to 300 amino acids, from 300 amino acids to 350 amino acids, from 350 amino acids to 400 amino acids, from 400 amino acids to 450 amino acids, from amino acids to 500 amino acids, from 500 amino acids to 550 amino acids, from 550 amino acids to 600 amino acids, from 600 amino acids to 650 amino acids, from 650 amino acids to 700 amino acids, from 700 amino acids to 750 amino acids, from 750 amino acids to 800 amino acids, or from 800 amino acids to 847 amino acids) of the CD22 amino acid sequence depicted in FIG. 7. As another example, a CAA present in a CT-MP comprises an amino acid sequence having at least 80%, at least 85%, at least 90%, at least 95%, at least 98%, at least 99%, or 100%, amino acid sequence identity to a contiguous stretch of from 25 amino acids to 310 amino acids (e.g., from 25 amino acids to 50 amino acids, from 50 amino acids to 100 amino acids, from 100 amino acids to 150 amino acids, from 150 amino acids to 200 amino acids, from 200 amino acids to 250 amino acids, from 250 amino acids to 300 amino acids, or from 300 amino acids to 310 amino acids) of the following amino acid sequence:

(SEQ ID NO: 5)
DSSKWVFEHPETLYAWEGACVWIPCTYRALDGDLESFILFHNPEYNKNT
SKFDGTRLYESTKDGKVPSEQKRVQFLGDKNKNCTLSIHPVHLNDSGQL
GLRMESKTEKWMERIHLNVSERPFPPHIQLPPEIQESQEVTLTCLLNFS
CYGYPIQLQWLLEGVPMRQAAVTSTSLTIKSVFTRSELKFSPQWSHHGK
IVTCQLQDADGKFLSNDTVQLNVKHTPKLEIKVTPSDAIVREGDSVTMT
CEVSSSNPEYTTVSWLKDGTSLKKQNTFTLNLREVTKDQSGKYCCQVSN
DVGPGRSEEVFLQVQYA.

### AFP CAR-BPs

In some cases, a CAA present in a CT-MP can comprise an amino acid sequence having at least 80%, at least 85%, at least 90%, at least 95%, at least 98%, at least 99%, or 100%, amino acid sequence identity to a contiguous stretch of from 25 amino acids to 609 amino acids (e.g., from 25 amino acids to 50 amino acids, from 50 amino acids to 100 amino acids, from 100 amino acids to 150 amino acids, from 150 amino acids to 200 amino acids, from 200 amino acids to 250 amino acids, from 250 amino acids to 300 amino acids, from 300 amino acids to 350 amino acids, from 350 amino acids to 400 amino acids, from 400 amino acids to 450 amino acids, from amino acids to 500 amino acids, from 500 amino acids to 550 amino acids, or from 550 amino acids to 609 amino acids) of the AFP amino acid sequence depicted in FIG. 10.

### MAGE-A4 CAR-BPs

As another example, a CAA present in a CT-MP can comprise an amino acid sequence having at least 80%, at least 85%, at least 90%, at least 95%, at least 98%, at least 99%, or 100%, amino acid sequence identity to a contiguous stretch of from 25 amino acids to 320 amino acids (e.g., from 25 amino acids to 50 amino acids, from 50 amino acids to 100 amino acids, from 100 amino acids to 150 amino acids, from 150 amino acids to 200 amino acids, from 200 amino acids to 250 amino acids, from 250 amino acids to 300 amino acids, or from 300 amino acids to 320 amino acids) of the following melanoma-associated antigen 4 (MAGE-A4) amino acid sequence:

(SEQ ID NO: 6)
MSSEQKSQHC KPEEGVEAQE EALGLVGAQA PTTEEQEAAV SSSSPLVPGT
LEEVPAAESA GPPQSPQGAS ALPTTISFTC WRQPNEGSSS QEEEGPSTSP DAESLFREAL
SNKVDELAHF LLRKYRAKEL VTKAEMLERV IKNYKRCFPV IFGKASESLK MIFGIDVKEV
DPASNTYTLV TCLGLSYDGL LGNNQIFPKT GLLIIVLGTI AMEGDSASEE EIWEELGVMG
VYDGREHTVY GEPRKLLTQD WVQENYLEYR QVPGSNPARY EFLWGPRALA ETSYVKVLEH
VVRVNARVRI AYPSLREAAL LEEEEGV.

### NY-ESO-1 CAR-BPs

As another example, a CAA present in a CT-MP is a Cancer/Testis Antigen-1 (CTAG1B) peptide. CTAG1B is also known as LAGE2, LAGE3, or NY-ESO-1 (New York Esophageal Squamous Cell Carcinoma 1). For example, in some cases, a CAA present in a CT-MP can comprise an amino acid sequence having at least 80%, at least 85%, at least 90%, at least 95%, at least 98%, at least 99%, or 100%, amino acid sequence identity to a contiguous stretch of from 25 amino acids to 180 amino acids (e.g., from 25 amino acids to 50 amino acids, from 50 amino acids to 100 amino acids, from 100 amino acids to 150 amino acids, or from 150 amino acids to 180 amino acids) of the following NY-ESO-1 amino acid sequence: MQAEGRGTGG STGDADGPGG PGIPDGPGGN AGGPGEAGAT GGRGPRGAGA ARASGPGGGA PRGPHGGAAS GLNGCCRCGA RGPESRLLEF YLAMPFATPM EAELARRSLA QDAPPLPVPG VLLKEFTVSG NILTIRLTAA DHRQLQLSIS SCLQQLSLLM WITQCFLPVF LAQPPSGQRR (SEQ ID NO:7).

### Mesothelin CAR-BPs

As another example, a CAA present in a CT-MP can comprise an amino acid sequence having at least 80%, at least 85%, at least 90%, at least 95%, at least 98%, at least 99%, or 100%, amino acid sequence identity to a contiguous stretch of from 25 amino acids to 600 amino acids (e.g., from 25 amino acids to 50 amino acids, from 50 amino acids to 100 amino acids, from 100 amino acids to 150 amino acids, from 150 amino acids to 200 amino acids, from 200 amino acids to 250 amino acids, from 250 amino acids to 300 amino acids, from 300 amino acids to 350 amino acids, from 350 amino acids to 400 amino acids, from 400 amino acids to 450 amino acids, from amino acids to 500 amino acids, from 500 amino acids to 550 amino acids, or from 550 amino acids to 600 amino acids) of the following mesothelin amino acid sequence:

(SEQ ID NO: 8)
MALPTARPLL GSCGTPALGS LLFLLFSLGW VQPSRTLAGE TGQEAAPLDG
VLANPPNISS LSPRQLLGFP CAEVSGLSTE RVRELAVALA QKNVKLSTEQ LRCLAHRLSE
PPEDLDALPL DLLLFLNPDA FSGPQACTRF FSRITKANVD LLPRGAPERQ RLLPAALACW
GVRGSLLSEA DVRALGGLAC DLPGRFVAES AEVLLPRLVS CPGPLDQDQQ EAARAALQGG
GPPYGPPSTW SVSTMDALRG LLPVLGQPII RSIPQGIVAA WRQRSSRDPS WRQPERTILR
PRFRREVEKT ACPSGKKARE IDESLIFYKK WELEACVDAA LLATQMDRVN AIPFTYEQLD
VLKHKLDELY PQGYPESVIQ HLGYLFLKMS PEDIRKWNVT SLETLKALLE VNKGHEMSPQ
VATLIDRFVK GRGQLDKDTL DTLTAFYPGY LCSLSPEELS SVPPSSIWAV RPQDLDTCDP
RQLDVLYPKA RLAFQNMNGS EYFVKIQSFL GGAPTEDLKA LSQQNVSMDL ATFMKLRTDA
VLPLTVAEVQ KLLGPHVEGL KAEERHRPVR DWILRQRQDD LDTLGLGLQG GIPNGYLVLD
LSMQEALSGT PCLLGPGPVL TVLALLLAST LA

### T-Cell Modulating Polypeptides (TMPs)

T cells have receptors, e.g., IL-2 receptor (IL-2R), that when engaged by an activating polypeptide (e.g., IL-2), can lead to proliferation and/or activation of the T cell. In some cases, a TMP present in a CT-MP is a wild-type (“wt”) TMP. In other cases, a TMP present in a CT-MP is a variant of a wt TMP that has reduced affinity for a cognate co-receptor (“co-TMP”) for the TMP, compared to the affinity of a corresponding wild-type TMP for the co-TMP. Suitable TMPs that exhibit reduced affinity for a co-TMP can have from 1 amino acid (aa) to 20 aa differences from a wild-type TMP. For example, in some cases, a variant TMP present in a CT-MP differs in amino acid sequence by 1 aa, 2 aa, 3 aa, 4 aa, 5 aa, 6 aa, 7 aa, 8 aa, 9 aa, or 10 aa, from a corresponding wild-type TMP. As another example, in some cases, a variant TMP present in a CT-MP differs in amino acid sequence by 11 aa, 12 aa, 13 aa, 14 aa, 15 aa, 16 aa, 17 aa, 18 aa, 19 aa, or 20 aa, from a corresponding wild-type TMP.

Although a TMP typically will be a polypeptide that cause activation and/or proliferation of a cell therapy T cell such as a CAR-T cell, in some cases a TMP can be a polypeptide (e.g., PD-L1 of Fas ligand (FasL) that causes suppression/inhibition of a T cell. Such suppressing/inhibitory TMPs could be employed, e.g., where the therapeutic goal is to reduce the activity of the target T cells.

A TMP also could be a polypeptide such as an antibody, a binding fragment of an antibody, or other ligand that binds to and blocks an immune checkpoint on the T cell such as PD-1, CTLA-4, TIGIT or LAG3. Such TMPs, which could reduce the potential of the T cell to be inhibited or suppressed due to an immune checkpoint. Such TMPs also could be employed in combination with a TMP that causes activation and/or proliferation of a cell therapy T cell such as a CAR-T cell, e.g., in a heterodimeric CT-MP comprising an interspecific dimerization sequence as discussed herein.

A TMP may comprise a variant of a wt TMP that may exhibit reduced binding to its co-TMP, including e.g., reduced binding to one or more chains or domains of the co-TMP. For example, a variant TMP present in a CT-MP may bind its co-TMP with an affinity that it at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the affinity of a corresponding wild-type TMP for the co-TMP.

Exemplary pairs of TMPs and their co-TMPs include, but are not limited to those set out in Table 1, below:

TABLE 1

| T-Cell Modulating Polypeptide (TMP) | Co-TMP |
|---|---|
| 4-1BBL | 4-1BB |
| IL-2 | IL-2 receptor |
| CD80 | CD28 |
| CD86 | CD28 |
| OX40L (CD252) | OX40 (CD134) |
| ICOS-L | ICOS |
| PD-L1 | PD-1 |
| Fas ligand (FasL) | Fas |
| Anti-PD1 | PD-1 |
| Anti-TIGIT | TIGIT |
| Anti-LAG3 | LAG3 |
| Anti-CTLA-4 | CTLA-4 |

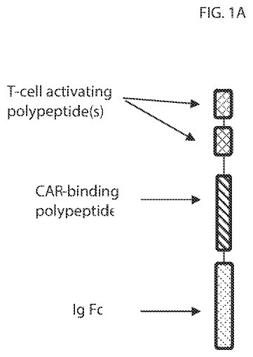

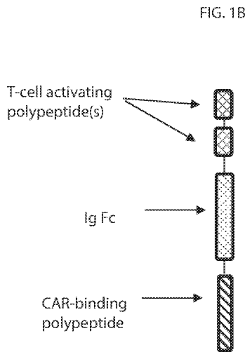

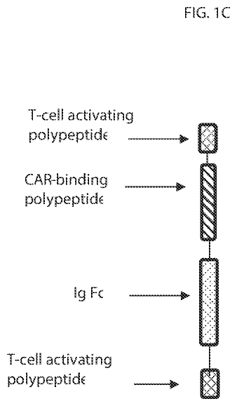

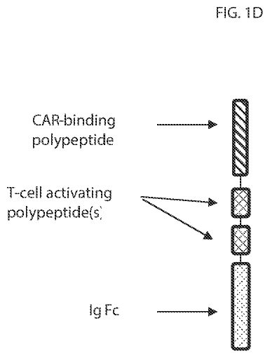

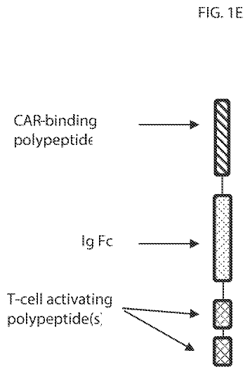

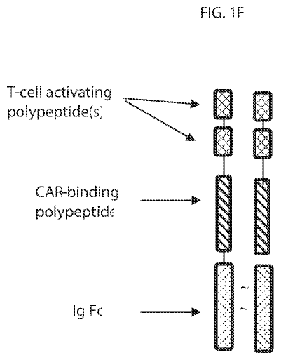

As depicted schematically in FIG. 1A-1E, one or more TMPs can be present in a CAR-T MP at any of a variety of positions. FIG. 1A-1E depicts the position of two TMPs; however, the TMP can be any number of and any of a variety of TMPs, as described herein. As depicted in FIG. 1A, a TMP can be at the N-terminus of the CT-MP. As depicted in FIG. 1C, TMPs can be located at both the N-terminus of the CT-MP and at the C-terminus of the CT-MP. As depicted in FIG. 1D, a TMP can be C-terminal to a CAR-BP and N-terminal to an Ig Fc polypeptide (i.e., the TMP can be positioned between the CAR-BP and the Ig Fc polypeptide). As depicted in FIG. 1E, a TMP can be at the C-terminus of a CT-MP.

Wild-type TMPs and variants, including reduced affinity variants, such as IL-2, 4-1BBL, CD80, and CD86 are described in the published literature, e.g., published PCT application WO2020132138A1 and WO2019/051091, the disclosures of which as they pertain to wild-type and specific variants of IL-2, 4-1BBL, CD80, and CD86 are expressly incorporated herein by reference.

Of specific interest are TMPs that are variants of the cytokine IL-2. Wild-type IL-2 binds to IL-2 receptor (IL-2R) on the surface of a T cell. Wild-type IL-2 has a strong affinity for IL-2R and will bind to activate most or substantially all CD8+ T cells. For this reason, synthetic forms of wild type IL-2 such as the drug aldesleukin (trade name Proleukin®) are known to have severe side-effects when administered to humans for the treatment of cancer because the IL-2 indiscriminately activates both target and non-target T cells.

An IL-2R is in some cases a heterotrimeric polypeptide comprising an alpha chain (IL-2Rα; also referred to as CD25), a beta chain (IL-2Rβ; also referred to as CD122; and a gamma chain (IL-2Rγ; also referred to as CD132). Amino acid sequences of human IL-2, human IL-2Rα, IL2Rβ, and IL-2Rγ are known. See, e.g., published PCT applications WO2020132138A1 and WO2019/051091, discussed above.

In some cases, an IL-2 variant polypeptide present in a CT-MP of this disclosure exhibits decreased binding to IL-2Rα, thereby minimizing or substantially reducing the activation of Tregs by the IL-2 variant. Alternatively, or additionally, in some cases, an IL-2 variant polypeptide present in a CT-MP of this disclosure exhibits decreased binding to IL-2Rβ and/or IL-2Rγ such that the IL-2 variant polypeptide exhibits an overall reduced affinity for IL-2R. In some cases, an IL-2 variant polypeptide present in a CT-MP of this disclosure exhibits both properties, i.e., it exhibits decreased or substantially no binding to IL-2Rα, and also exhibits decreased binding to IL-2Rβ, IL-2Rγ, or both IL-2β and IL-2Rγ such that the IL-2 variant polypeptide exhibits an overall reduced affinity for IL-2R. For example, IL-2 variants having substitutions at H16 and F42 have shown decreased binding to IL-2Rα and IL-2Rβ. See, Quayle et al., Clin Cancer Res; 26(8) Apr. 15, 2020, which discloses that the binding affinity of an IL-2 polypeptide with H16A and F42A substitutions for human IL-2Rα and IL-2Rβ was decreased 110- and 3-fold, respectively, compared with wild-type IL2 binding, predominantly due to a faster off-rate for each of these interactions. CT-MPs comprising such variants, including variants that exhibit decreased binding to IL-2Rα and IL-2Rβ, are less likely to deliver IL-2 to non-target T cells, i.e., T cells that do not contain a CAR. That is, the binding of the variant IL-2 TMP to IL-2R on T cells in vivo or in vitro is more likely to occur when there binding of the CAR-BP to the CAR of the T cell than when the CAR-BP of the CT-MP is not bound to a CAR on a T cell.

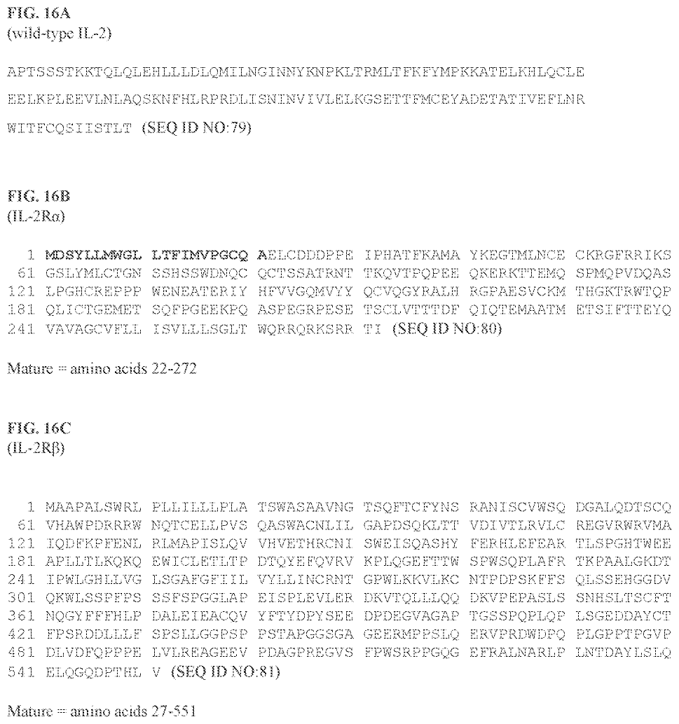

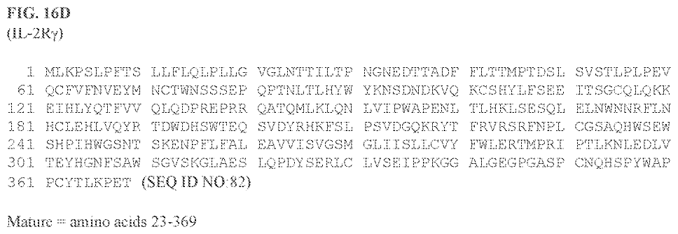

Suitable IL-2 variant polypeptides thus include a polypeptide that comprises an amino acid sequence having at least 90%, at least 95%, at least 98%, or at least 99% amino acid sequence identity to the wild-type IL-2 amino acid sequence depicted in FIG. 16A; and that have one or more amino acid differences from the wild-type IL-2 amino acid sequence depicted in FIG. 16A. In some cases, such a variant IL-2 polypeptide of this disclosure exhibits reduced binding affinity to IL-2R, compared to the binding affinity of an IL-2 polypeptide comprising the wild-type IL-2 amino acid sequence depicted in FIG. 16A. For example, in some cases, a variant IL-2 polypeptide binds IL-2R with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of an IL-2 polypeptide comprising the wild-type IL-2 amino acid sequence depicted in FIG. 16A for an IL-2R (e.g., an IL-2R comprising polypeptides comprising the amino acid sequences depicted in FIG. 16B-16D), when assayed under the same conditions.

In some cases, a suitable variant IL-2 polypeptide comprises an amino acid sequence having at least 90%, at least 95%, at least 98%, at least 99%, or 100% amino acid sequence identity to the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO: 9), i.e., the variant IL-2 polypeptide has the amino acid sequence of wild-type IL-2 but with H16A and F42A substitutions (shown in bold). Alternatively, the foregoing sequence, but with substitutions other than Ala at H16 and/or F42 may be employed, e.g., H16T may be employed instead of H16A. In some cases, a variant IL-2 polypeptide present in a CT-MP comprises the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO: 9). In some cases, a variant IL-2 polypeptide present in a CT-MP comprises the amino acid sequence: APTSSSTKKT QLQLETLLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:10). In some cases, a CT-MP comprises two copies of such a variant IL-2 polypeptide.

In some cases, a TMP present in a CT-MP is a 4-1BBL polypeptide. In some cases, a 4-1BBL polypeptide of a CT-MP comprises an amino acid sequence having at least 75%, at least 80%, at least 85%, at least 90%, at least 95%, at least 98%, at least 99%, or 100%, amino acid sequence identity to the following 4-1BBL amino acid sequence: DPAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPA (SEQ ID NO:11).

In some cases, a TMP present in a CT-MP is an ICOS-L polypeptide. In some cases, an ICOS-L polypeptide of a CT-MP comprises an amino acid sequence having at least 75%, at least 80%, at least 85%, at least 90%, at least 95%, at least 98%, at least 99%, or 100%, amino acid sequence identity to the following ICOS-L amino acid sequence: QEKEVRAMVG SDVELSCACP EGSRFDLNDV YVYWQTSESK TVVTYHIPQN SSLENVDSRY RNRALMSPAG MLRGDFSLRL FNVTPQDEQK FHCLVLSQSL GFQEVLSVEV TLHVAANFSV PVVSAPHSPS QDELTFTCTS INGYPRPNVY WINKTDNSLL DQALQNDTVF LNMRGLYDVV SVLRIARTPS VNIGCCIENV LLQQNLTVGS QTGNDIGERD KITENPVSTG EKNAATWSIL (SEQ ID NO:12).

In some cases, a TMP present in a CT-MP is an OX40L polypeptide. In some cases, an OX40L polypeptide of a CT-MP comprises an amino acid sequence having at least 75%, at least 80%, at least 85%, at least 90%, at least 95%, at least 98%, at least 99%, or 100%, amino acid sequence identity to the following OX40L amino acid sequence: L QVSHRYPRIQ SIKVQFTEYK KEKGFILTSQ KEDEIMKVQN NSVIINCDGF YLISLKGYFS QEVNISLHYQ KDEEPLFQLK KVRSVNSLMV ASLTYKDKVY LNVTTDNTSL DDFHVNGGEL ILIHQNPGEF CVL (SEQ ID NO:13).

In some cases, a TMP present in a CT-MP is a CD80 polypeptide. In some cases, a CD80 polypeptide of a CT-MP comprises an amino acid sequence having at least 75%, at least 80%, at least 85%, at least 90%, at least 95%, at least 98%, at least 99%, or 100%, amino acid sequence identity to following CD80 amino acid sequence: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: (SEQ ID NO:14).

In some cases, a TMP present in a CT-MP is a CD86 polypeptide. In some cases, a CD86 polypeptide of a CT-MP comprises an amino acid sequence having at least 75%, at least 80%, at least 85%, at least 90%, at least 95%, at least 98%, at least 99%, or 100%, amino acid sequence identity to the following CD86 amino acid sequence:

(SEQ ID NO: 15)
APLKIQAYFNETADLPCQFANSQNQSLSELVVFWQDQENLVLNEVYLGK
EKFDSVHSKYMNRTSFDSDSWTLRLHNLQIKDKGLYQCIIHHKKPTGMI
RIHQMNSELSVLANFSQPEIVPISNITENVYINLTCSSIHGYPEPKKMS
VLLRTKNSTIEYDGIMQKSQDNVTELYDVSISLSVSFPDVTSNMTIFCI
LETDKTRLLSSPFSIELEDPQPPPDHIP.

### Scaffold Component

A CT-MP comprises a scaffold component such as an Ig Fc polypeptide or other suitable polypeptide, or a carrier that can display both a CAR-BP and TMP.

Suitable polypeptides for use as a scaffold include antibody-based scaffold polypeptides and non-antibody-based scaffolds. Non-antibody-based scaffolds include, e.g., albumin, an XTEN (extended recombinant) polypeptide, transferrin, an Ig Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly; SEQ ID NO:84), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

Other suitable scaffold components that can display both a CAR-BP and TMP include carriers such as lipid vesicles (e.g., liposomes) or micelles, nanoparticles, PEGylated proteins (including site-specific PEGylation), fibronectin-based scaffold proteins, or artificial antigen presenting cells, such as engineered erythroid cells and enucleated cells (e.g., platelets).

Suitable scaffold polypeptides will in some cases be a half-life extending polypeptides. Thus, in some cases, a suitable scaffold polypeptide increases the in vivo half-life (e.g., the serum half-life) of the CT-MP, compared to a control CT-MP lacking the scaffold polypeptide. For example, in some cases, a scaffold polypeptide increases the in vivo half-life (e.g., the serum half-life) of the CT-MP, compared to a control CT-MP lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, an Ig Fc polypeptide increases the in vivo half-life (e.g., the serum half-life) of the CT-MP, compared to a control CT-MP lacking the Ig Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

As noted above, a CT-MP can comprise Ig Fc polypeptide. The Ig Fc polypeptide of a CT-MP can be a human IgG1 Fc, a human IgG2 Fc, a human IgG3 Fc, a human IgG4 Fc, etc., or a variant of a wild-type Ig Fc polypeptide. Variants include naturally-occurring variants, non-naturally-occurring variants, and combinations thereof.

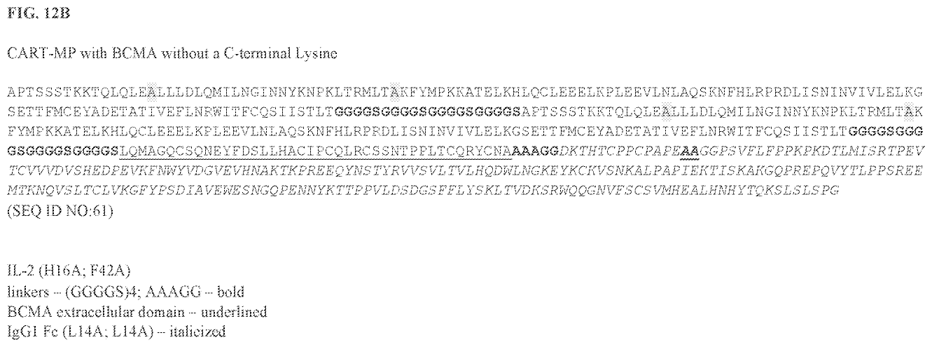

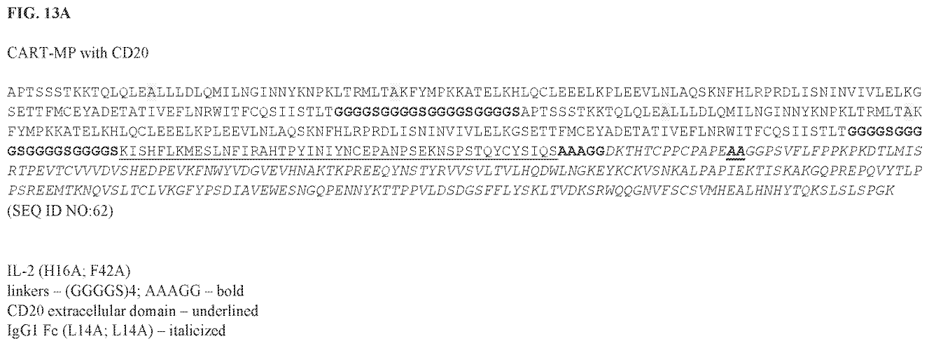

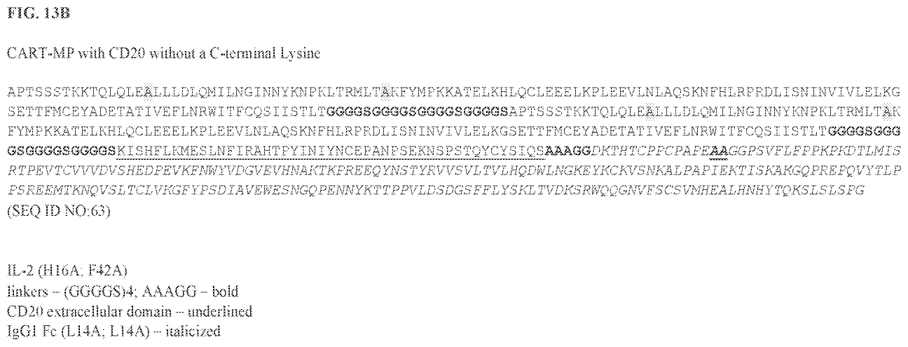

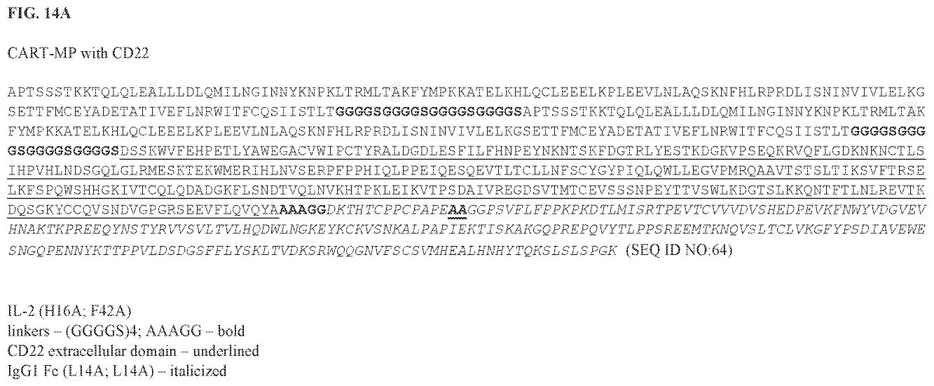

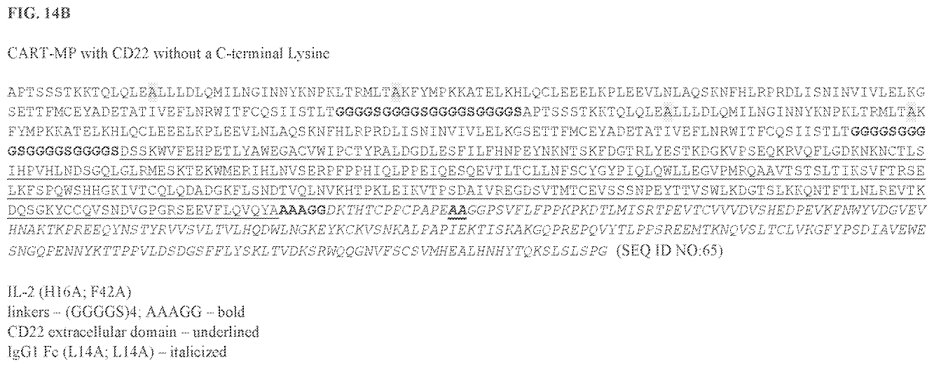

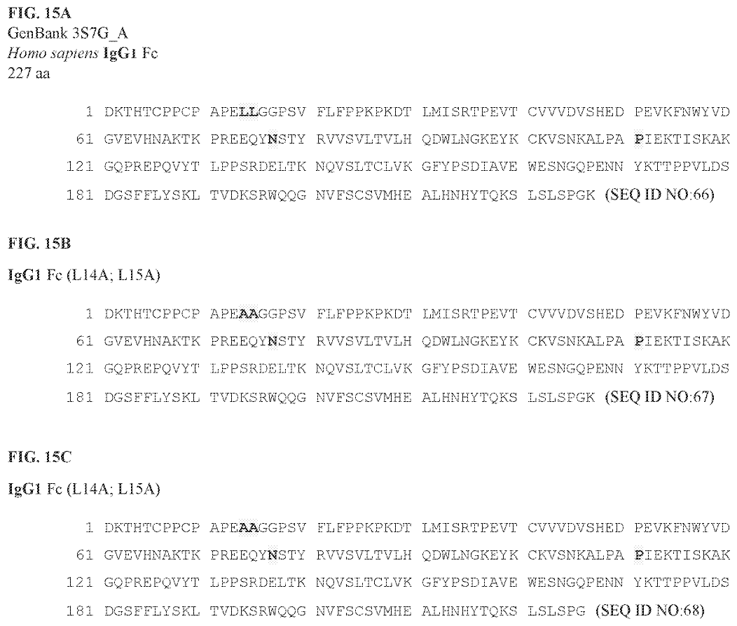

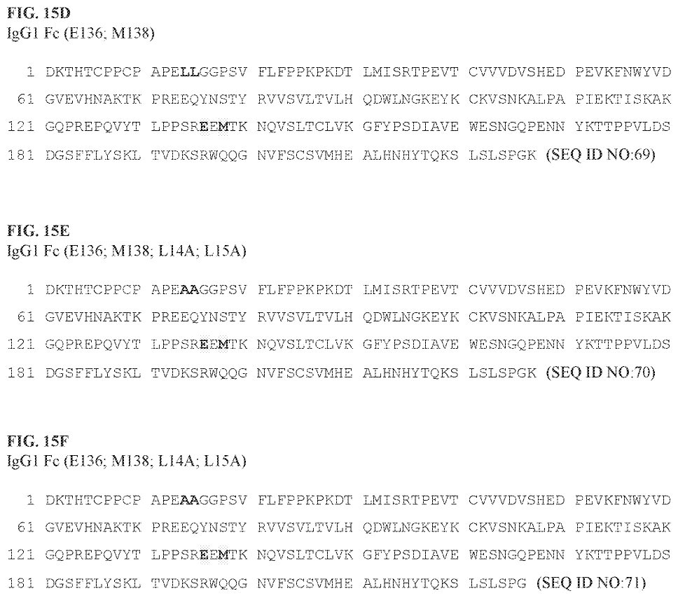

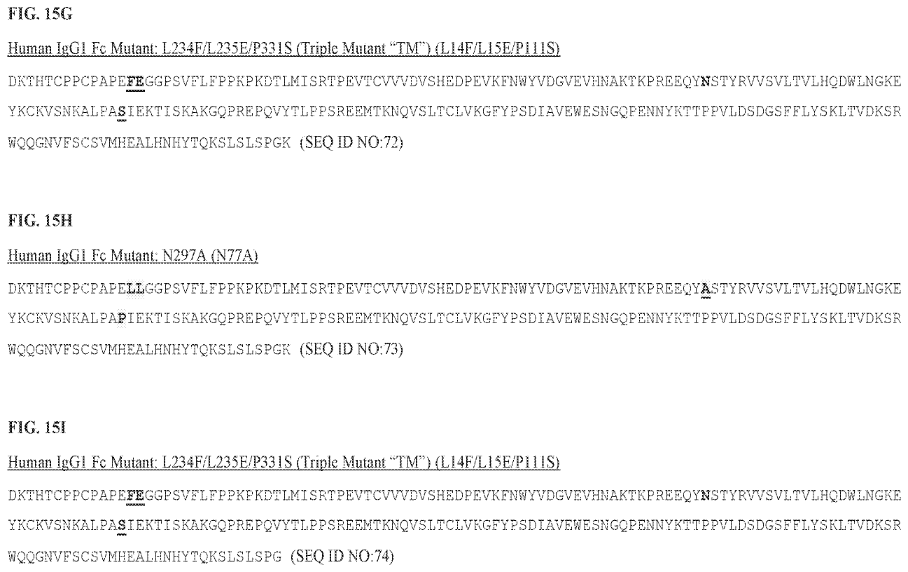

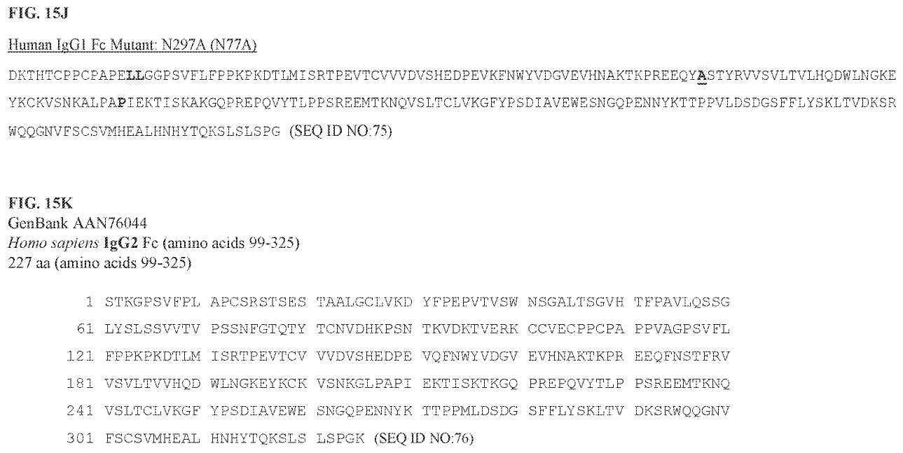

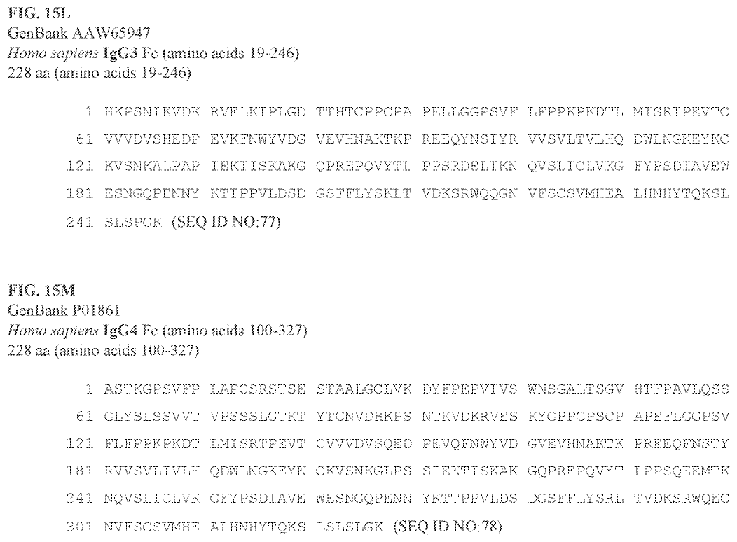

In some cases, the Ig Fc polypeptide present in a CT-MP may comprise an amino acid sequence having at least about 85%, at least about 90%, at least about 95%, at least about 98%, at least about 99%, or 100%, amino acid sequence identity to the Ig Fc amino acid sequence depicted in any one of FIG. 15A-15M. In embodiments, the Ig Fc polypeptide will be a variant that substantially does not induce cell lysis, e.g., through activation of complement-dependent cytotoxicity (CDC) and/or antibody-dependent cellular cytotoxicity (ADCC), and thus may include mutations that substantially reduce or eliminate the ability of the Ig polypeptide to induce cell lysis.

In some cases, the Ig Fc polypeptide present in a CT-MP is an IgG1 Fc polypeptide, or a variant of an IgG1 Fc polypeptide. For example, in some cases, the Ig Fc polypeptide present in a CT-MP comprises an amino acid sequence having at least about 85%, at least about 90%, at least about 95%, at least about 98%, at least about 99%, or 100%, amino acid sequence identity to the human IgG1 Fc polypeptide depicted in FIG. 15A. As another example, in some cases, the Ig Fc polypeptide present in a CT-MP comprises an amino acid sequence having at least about 85%, at least about 90%, at least about 95%, at least about 98%, at least about 99%, or 100%, amino acid sequence identity to the Ig Fc polypeptide depicted in FIG. 15B; where the Ig Fc polypeptide comprises an Ala at position 14 and an Ala at position 15. Such substitutions have been shown to reduce or substantially eliminate the ability of the Ig polypeptide to induce cell lysis. In any of the above embodiments, the Ig Fc polypeptide lacks a C-terminal lysine that is present in a wild-type IgG1 Fc polypeptide or a variant IgG1 Fc polypeptide. In any of the above embodiments, the Ig Fc polypeptide can have an N77 substitution; i.e., the Ig Fc polypeptide can have an amino acid other than Asn at position 77, where in some cases, the Ig Fc polypeptide has an Ala at position 77. In some cases, an Ig Fc polypeptide present in a CT-MP comprises the amino acid sequence depicted in FIG. 15A. In some cases, an Ig Fc polypeptide present in a CT-MP comprises the amino acid sequence depicted in FIG. 15B. In some cases, the IgG1 Fc polypeptide lacks a C-terminal lysine present in a wild-type IgG1 Fc polypeptide or a variant IgG1 Fc polypeptide. For example, in some cases, an Ig Fc polypeptide present in a CT-MP comprises the amino acid sequence depicted in FIG. 15C.

In some cases, the Ig Fc polypeptide present in a CT-MP is an IgG1 Fc polypeptide, or a variant of an IgG1 Fc polypeptide, where variants include naturally-occurring variants, non-naturally-occurring variants, and combinations thereof. For example, in some cases, the Ig Fc polypeptide present in a CT-MP comprises an amino acid sequence having at least about 85%, at least about 90%, at least about 95%, at least about 98%, at least about 99%, or 100%, amino acid sequence identity to the human IgG1 Fc polypeptide depicted in FIG. 15D; where the Ig Fc polypeptide comprises a Glu at position 136 and a Met at position 138. As another example, in some cases, the Ig Fc polypeptide present in a CT-MP comprises an amino acid sequence having at least about 85%, at least about 90%, at least about 95%, at least about 98%, at least about 99%, or 100%, amino acid sequence identity to the human IgG1 Fc polypeptide depicted in FIG. 15E; where the Ig Fc polypeptide has Ala at positions 14 and 15; and where the Ig Fc polypeptide comprises a Glu at position 136 and a Met at position 138. In any of the above embodiments, the Ig Fc polypeptide can have an N77 substitution; i.e., the Ig Fc polypeptide can have an amino acid other than Asn at position 77, where in some cases, the Ig Fc polypeptide has an Ala at position 77. In some cases, an Ig Fc polypeptide present in a CT-MP comprises the amino acid sequence depicted in FIG. 15D. In some cases, an Ig Fc polypeptide present in a CT-MP comprises the amino acid sequence depicted in FIG. 15E. In some cases, the IgG1 Fc polypeptide lacks a C-terminal lysine present in a wild-type IgG1 Fc polypeptide or a variant IgG1 Fc polypeptide. For example, in some cases, an Ig Fc polypeptide present in a CT-MP comprises the amino acid sequence depicted in FIG. 15F.

In some cases, the Ig Fc polypeptide present in a CT-MP comprises the amino acid sequence depicted in FIG. 15G (human IgG1 Fc comprising an L234F substitution, an L235E substitution, and a P331S substitution; where L234 corresponds to amino acid 14 of the amino acid sequence depicted in FIG. 15A or FIG. 15G; L235 corresponds to amino acid 15 of the amino acid sequence depicted in FIG. 15A or FIG. 15G; and P331 corresponds to amino acid 111 of the amino acid sequence depicted in FIG. 15A or FIG. 15G). In some cases, the Ig Fc polypeptide present in a CT-MP comprises the amino acid sequence depicted in FIG. 15H, comprising an N279A substitution (N77A of the amino acid sequence depicted in FIG. 15H). In some cases, the IgG1 Fc polypeptide lacks a C-terminal lysine present in a wild-type IgG1 Fc polypeptide or a variant IgG1 Fc polypeptide. For example, in some cases, an Ig Fc polypeptide present in a CT-MP comprises the amino acid sequence depicted in FIG. 15I or FIG. 15J.

In some cases, the Ig Fc polypeptide comprises an amino acid sequence having at least about 85%, at least about 90%, at least about 95%, at least about 98%, at least about 99%, or 100%, amino acid sequence identity to the human IgG2 Fc polypeptide depicted in FIG. 15K; e.g., the Ig Fc polypeptide comprises an amino acid sequence having at least about 85%, at least about 90%, at least about 95%, at least about 98%, at least about 99%, or 100%, amino acid sequence identity to amino acids 99-325 of the human IgG2 Fc polypeptide depicted in FIG. 15K (e.g., where the Ig Fc polypeptide has a length of about 227 amino acids). In some cases, the Ig Fc polypeptide comprises an amino acid sequence having at least about 85%, at least about 90%, at least about 95%, at least about 98%, at least about 99%, or 100%, amino acid sequence identity to the human IgG3 Fc polypeptide depicted in FIG. 15L; e.g., the Ig Fc polypeptide comprises an amino acid sequence having at least about 85%, at least about 90%, at least about 95%, at least about 98%, at least about 99%, or 100%, amino acid sequence identity to amino acids 19-246 of the human IgG3 Fc polypeptide depicted in FIG. 15L (e.g., where the Ig Fc polypeptide has a length of about 228 amino acids).

In some cases, the Ig Fc polypeptide comprises an amino acid sequence having at least about 85%, at least about 90%, at least about 95%, at least about 98%, at least about 99%, or 100%, amino acid sequence identity to the human IgG4 Fc polypeptide depicted in FIG. 15M. In some cases, the Ig Fc polypeptide comprises an amino acid sequence having at least about 85%, at least about 90%, at least about 95%, at least about 98%, at least about 99%, or 100%, amino acid sequence identity to amino acids 100 to 327 of the human IgG4 Fc polypeptide depicted in FIG. 15M (e.g., where the Ig Fc polypeptide has a length of about 228 amino acids).

In some cases, the Ig Fc employed in a CT-MP will comprise one or more substitutions of amino acids in the wild-type sequence, such that that Ig Fc that substantially does not induce cell lysis through Antibody-Dependent Cellular Cytotoxicity (“ADCC”) and/or Complement-Dependent Cytotoxicity (“CDC”). For example, in some cases the Ig Fc polypeptide present in a CT-MP comprises the amino acid sequence depicted in FIG. 15A (human IgG1 Fc), except for a substitution of L234 (L14 of the amino acid sequence depicted in FIG. 15A) with an amino acid other than leucine, and a substitution of L235 (L15 of the amino acid sequence depicted in FIG. 15A) with an amino acid other than leucine. Examples thus include an IgG1 Fc comprising an L234A (L14A) substitution and an L235A (L15A) substitution (see FIGS. 15B and C).

### Linkers

A CT-MP can include one or more independently selected peptide linkers, i.e., a linker comprising a contiguous stretch of two or more amino acids, where the one or more linkers are between one or more components of a CT-MP. For example, a CT-MP can include one or more independently selected peptide linkers, i.e., a linker comprising a contiguous stretch of two or more amino acids, where the one or more linkers are the same or different and are between one or more of: i) a TMP and a CAR-BP; ii) a CAR-BP and an Ig Fc polypeptide or other scaffold polypeptide; and iii) a TMP and an Ig Fc polypeptide. Where the CT-MP comprises more than one TMP in tandem, each TMP may be joined to the adjacent TMP by a linker.

As used herein, the phrase “an optional peptide linker between any two of the components of a CT-MP” refers to a peptide linker between any two adjacent polypeptides within the CT-MP. For example, as used herein, the phrase “an optional peptide linker between any two of the components of a CT-MP” refers to a peptide linker between one or more of: i) a TMP and a CAR-BP; ii) a CAR-BP and an Ig Fc polypeptide; iii) a first TMP and a second TMP; and iv) a TMP and an Ig Fc polypeptide. As discussed below, linkers may be: a) a flexible peptide linker, including a short flexible peptide linker; or b) a rigid peptide linker.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. In some cases, a linker has a length of from 25 amino acids to 50 amino acids, e.g., from 25 to 30, from 30 to 35, from 35 to 40, from 40 to 45, or from 45 to 50 amino acids in length.

### Flexible Peptide Linkers

Exemplary flexible peptide linkers include glycine polymers (G)ₙ, glycine-serine polymers (including, for example, (GS)ₙ, (GSGGS)ₙ (SEQ ID NO:16), (GGGGS)ₙ (SEQ ID NO:17), and (GGGS)ₙ (SEQ ID NO:83), where n is an integer of at least one and can be an integer from 1 to 10), glycine-alanine polymers, alanine-serine polymers, and other flexible peptide linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:18), GGSGG (SEQ ID NO: 19), GSGSG (SEQ ID NO:20), GSGGG (SEQ ID NO:21), GGGSG (SEQ ID NO:22), GSSSG (SEQ ID NO:23), and the like.

Exemplary flexible peptide linkers include, e.g., (GGGGS)n (SEQ ID NO:17); also referred to as a “G4S” linker), where n is 1, 2, 3, 4, 5, 6, 7, 8, 9, or 10. In some cases, a linker comprises the amino acid sequence (GGGGS)n (SEQ ID NO:17), where n is 1, 2, 3, 4, 5, 6, 7, 8, 9 or 10. In some cases, a linker comprises the amino acid sequence (GGGGS)n (SEQ ID NO:17), where n is 2. In some cases, a linker comprises the amino acid sequence (GGGGS)n (SEQ ID NO:17), where n is 3. In some cases, a linker comprises the amino acid sequence (GGGGS)n (SEQ ID NO:17), where n is 4. In some cases, a linker comprises the amino acid sequence (GGGGS)n (SEQ ID NO:17), where n is 7. In some cases, a linker comprises the amino acid sequence AAAGG (SEQ ID NO:24). Also suitable is a linker having the amino acid sequence AAAGG (SEQ ID NO:24).

As used in this disclosure, a “short flexible peptide linker” means a flexible peptide linker that comprises fewer than 15 amino acids, i.e., from 2-14 amino acids. For example, a short flexible peptide linker can comprise from 2-4 amino acids, from 2-5 amino acids, from 3-6 amino acids, from 4-8 amino acids, from 5-10 amino acids, or from 10-14 amino acids. Within this range includes flexible peptide linkers comprising 2, 3, 4, 5, 6, 7, 8, 9, 10, 11, 12, 13 or 14 amino acids.

### Rigid Peptide Linkers

In some cases, a peptide linker is a rigid peptide linker. As used herein, the term “rigid peptide linker” refers to a linker comprising a contiguous stretch of two or more amino acids that effectively separates protein domains by maintaining a substantially fixed distance/spatial separation between the domains, thereby reducing or substantially eliminating unfavorable interactions between such domains. For example, rigid linkers may be interposed when either the TMP(s) or the CAR-BP are located in the N-terminus and/or the C-terminus of the CT-MP. Rigid peptide linkers are known in the art and generally adopt a relatively well-defined conformation when in solution. Rigid peptide linkers include those which have a particular secondary and/or tertiary structure in solution; and are typically of a length sufficient to confer secondary or tertiary structure to the linker. Rigid peptide linkers include peptide linkers rich in proline, and peptide linkers having an inflexible helical structure, such as an α-helical structure. Rigid peptide linkers are described in, for example, Chen et al. (2013) Adv. Drug Deliv. Rev. 65:1357; and Klein et al. (2014) Protein Engineering, Design & Selection 27:325.

Examples of rigid peptide linkers include, e.g., (EAAAK)n (SEQ ID NO:25), A(EAAAK)nA (SEQ ID NO:26), A(EAAAK)nALEA(EAAAK)nA (SEQ ID NO:27), (Lys-Pro)n, (Glu-Pro)n, (Thr-Pro-Arg)n, and (Ala-Pro)n where n is an integer from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid peptide linkers comprising EAAAK (SEQ ID NO:28) include EAAAK (SEQ ID NO:28), (EAAAK)₂ (SEQ ID NO:29), (EAAAK)₃ (SEQ ID NO:30), A(EAAAK)₄ALEA(EAAAK)₄A (SEQ ID NO:31), and AEAAAKEAAAKA (SEQ ID NO:32). Non-limiting examples of suitable rigid peptide linkers comprising (AP)n include PAPAP (SEQ ID NO:33; also referred to herein as “(AP)2”); APAPAPAP (SEQ ID NO:34; also referred to herein as “(AP)4”); APAPAPAPAPAP (SEQ ID NO:35; also referred to herein as “(AP)6”); APAPAPAPAPAPAPAP (SEQ ID NO:36; also referred to herein as “(AP)8”); and APAPAPAPAPAPAPAPAPAP (SEQ ID NO:37; also referred to herein as “(AP)10”). Non-limiting examples of suitable rigid peptide linkers comprising (KP)n include KPKP (SEQ ID NO:38; also referred to herein as “(KP)2”); KPKPKPKP (SEQ ID NO:39; also referred to herein as “(KP)4”); KPKPKPKPKPKP (SEQ ID NO:40; also referred to herein as “(KP)6”); KPKPKPKPKPKPKPKP (SEQ ID NO:41; also referred to herein as “(KP)8”); and KPKPKPKPKPKPKPKPKPKP (SEQ ID NO:42; also referred to herein as “(KP)10”). Non-limiting examples of suitable rigid peptide linkers comprising (EP)n include EPEP (SEQ ID NO:43; also referred to herein as “(EP)2”); EPEPEPEP (SEQ ID NO:44; also referred to herein as “(EP)4”); EPEPEPEPEPEP (SEQ ID NO:45; also referred to herein as “(EP)6”); EPEPEPEPEPEPEPEP (SEQ ID NO:46; also referred to herein as “(EP)8”); and EPEPEPEPEPEPEPEPEPEP (SEQ ID NO:47; also referred to herein as “(EP)10”).

### Exemplary CT-MPs

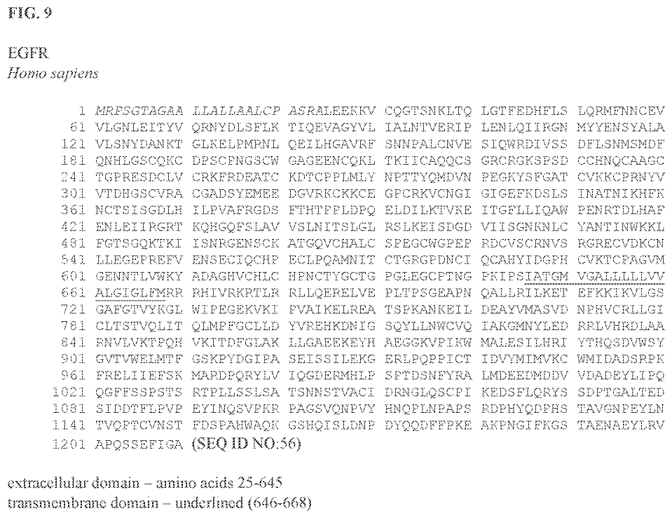

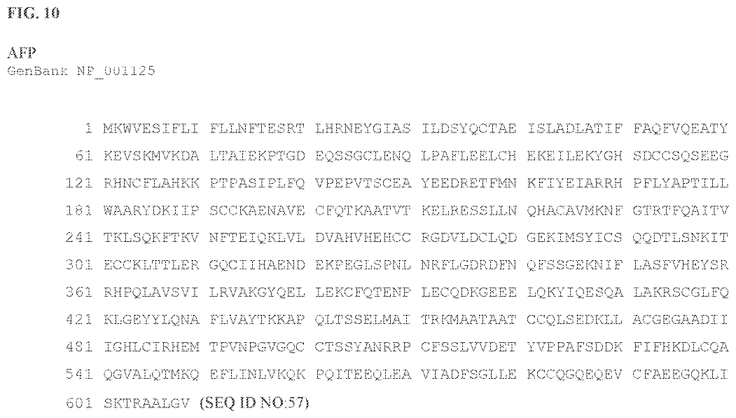

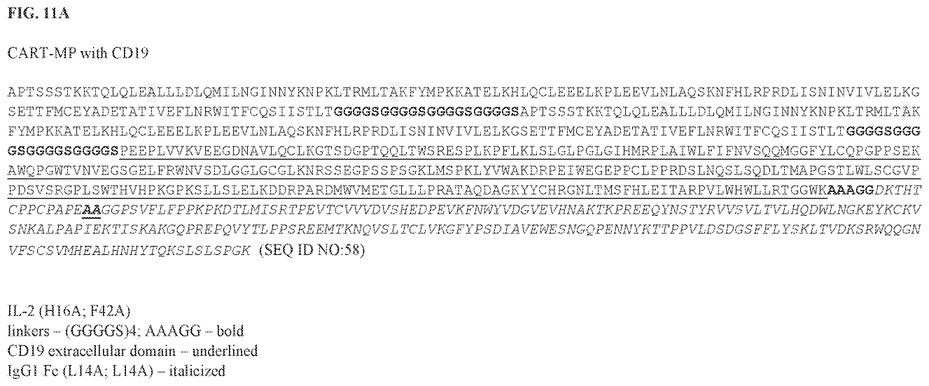

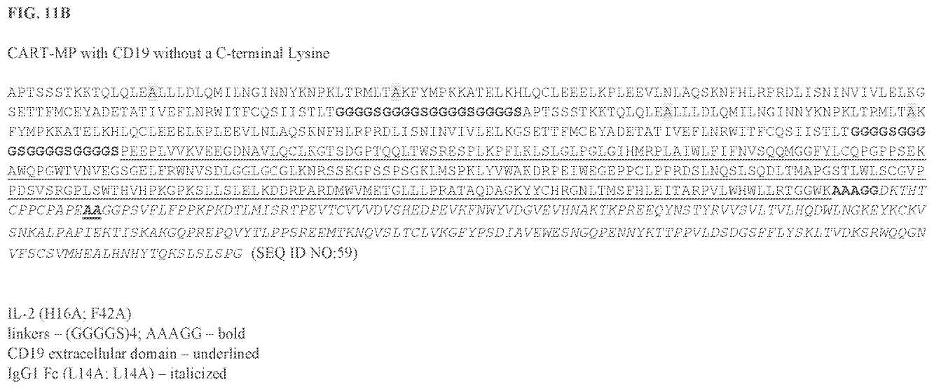

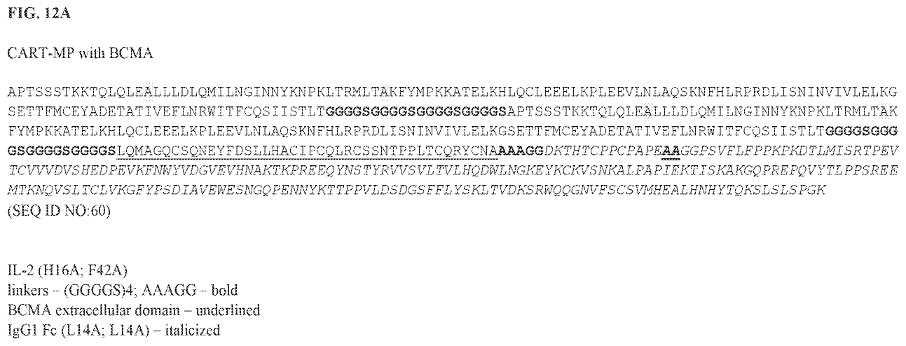

Exemplary CT-MPs are illustrated in FIGS. 11-14. FIGS. 11A and B provide an exemplary CT-MP comprising a CD19 extracellular domain, with and without a C-terminal lysine on the IgG1 Fc. FIGS. 12A and B provide an exemplary CT-MP comprising a BCMA extracellular domain, with and without a C-terminal lysine on the IgG1 Fc. FIGS. 13A and B provide an exemplary CT-MP comprising a CD20 extracellular domain, with and without a C-terminal lysine on the IgG1 Fc. FIGS. 14A and B provide an exemplary CT-MP comprising a CD22 extracellular domain, with and without a C-terminal lysine on the IgG1 Fc.

### Dimerized CT-MPs

A CT-MP can be dimerized; i.e., this disclosure provides a dimeric (homodimeric or heterodimeric) polypeptide comprising a dimer of a CT-MP. In some cases, the two CT-MPs of a dimeric polypeptide are identical to one another in amino acid sequence; i.e., the dimer is a homodimer. A dimeric polypeptide can comprise one or more disulfide bonds between the Ig Fc polypeptides of two CT-MPs. An example of such a dimeric polypeptide is depicted schematically in FIG. 1F, where “-” represents a disulfide bond.

Alternatively, in some cases, the two CT-MPs of a dimeric polypeptide are not identical to one another in amino acid sequence, i.e., the dimer is a heterodimer. In such cases, for example, the Ig Fc polypeptides of each CT-MP can comprise interspecific dimerization sequences, e.g., “Knob-in-Hole” sequences that permit two different CT-MP s to selectively dimerize. Interspecific binding sequences favor formation of heterodimers with their cognate polypeptide sequence (i.e., the interspecific sequence and its counterpart interspecific sequence), particularly those based on Ig Fc sequence variants. Such interspecific polypeptide sequences include Knob-in-Hole, and Knob-in-Hole sequences that facilitate the formation of one or more disulfide bonds. For example, one interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequence. See Xu et al. mAbs 7:1, 231-242 (2015). Another interspecific binding pair has a first Fc polypeptide with Y349C, T366S, L368A, and Y407V substitutions and a second Ig Fc polypeptide with S354C, and T366W substitutions (disulfide bonds can form between the Y349C and the S354C). See, e.g., Brinkmann and Konthermann, mAbs 9:2, 182-212 (2015). Ig Fc polypeptide sequences, either with or without knob-in-hole modifications, can be stabilized by the formation of disulfide bonds between the Ig Fc polypeptides (e.g., the hinge region disulfide bonds). Thus, in some cases, a dimerized CT-MP can be a heterodimer, comprising two CT-MP chains that are not identical in amino acid sequence. Interspecific sequences can be used, e.g., where it is desired to provide a CT-MP with two different TMPs. For example, dimeric CT-MPs could comprise 4-1BBL and CD80, IL-2 and CD86, or IL-2 and CD80 as the TMPs, or combinations of a TMP that causes activation and/or proliferation together with a TMP that binds to and inhibits an immune checkpoint from binding to a ligand on a cancer cell that would suppress/inhibit the CT-MP.

Interspecific dimerization sequences also may be employed to enable TMPs to be linked to non-CT-MP molecules that can provide additional functionality to the CT-MP.

### Compositions

This disclosure provides a composition comprising a CT-MP, including a pharmaceutical composition comprising a CT-MP.

The composition may comprise one or more pharmaceutically acceptable additives, a variety of which are known in the art and need not be discussed in detail herein. See, for example, the ninth (or latest) edition of Sheskey et al., “Handbook of Pharmaceutical Excipients” (2020), and/or the 23^{rd} (or latest) edition of “Remington: The Science and Practice of Pharmacy”, 23rd Ed. (2020).

In some cases, a subject pharmaceutical composition will be suitable for administration to a subject, e.g., will be sterile. For example, in some cases, a subject pharmaceutical composition will be suitable for administration to a human subject, e.g., where the composition is sterile and is substantially free of detectable pyrogens and/or other toxins, or where such detectable pyrogens and/or other toxins are present at a level within acceptable limits set by an applicable regulatory agency, e.g., the USF&DA.

For example, compositions may include aqueous solution, powder form, granules, tablets, pills, suppositories, capsules, suspensions, sprays, and the like. The composition may be formulated according to the various routes of administration described below.

Where a CT-MP is administered as an injectable (e.g. subcutaneously, intraperitoneally, intramuscularly, and/or intravenously) directly into a tissue, a formulation can be provided as a ready-to-use dosage form, or as non-aqueous form (e.g. a reconstitutable storage-stable powder) or aqueous form, such as liquid composed of pharmaceutically acceptable carriers and excipients. The protein-containing formulations may also be provided so as to enhance serum half-life of the CT-MP following administration. For example, the CT-MP may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

Other examples of formulations suitable for parenteral administration include isotonic sterile injection solutions, anti-oxidants, bacteriostats, and solutes that render the formulation isotonic with the blood of the intended recipient, suspending agents, solubilizers, thickening agents, stabilizers, and preservatives. For example, a subject pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of a CT-MP in a formulation can vary widely (e.g., from less than about 0.1%, usually at or at least about 2% to as much as 20% to 50% or more by weight) and will usually be selected primarily based on fluid volumes, viscosities, and patient-based factors in accordance with the particular mode of administration selected and the patient's needs.

This disclosure provides a container comprising a composition comprising a CT-MP or pharmaceutical composition comprising a CT-MP, e.g., a liquid composition. The container can be, e.g., a syringe, an ampoule, and the like. In some cases, the container is sterile. In some cases, both the container and the composition are sterile.

In some cases, a CT-MP is present in a liquid composition. Thus, this disclosure provides compositions (e.g., liquid compositions, including pharmaceutical compositions) comprising a CT-MP. In some cases, a composition comprises: a) a CT-MP; and b) saline (e.g., 0.9% NaCl). In some cases, the composition is sterile. In some cases, the composition is suitable for administration to a human subject, e.g., where the composition is sterile and is free of detectable pyrogens and/or other toxins. Thus, this disclosure provides a composition comprising: a) a CT-MP; and b) saline (e.g., 0.9% NaCl), where the composition is sterile and is free of detectable pyrogens and/or other toxins.

### Nucleic Acids, Recombinant Expression Vectors, and Genetically Modified Host Cells

The present disclosure provides a nucleic acid comprising a nucleotide sequence encoding one or more polypeptides of a CT-MP or higher order CT-MP complex such as a dimer. Where two or more nucleotide sequences encode two or more polypeptides of a CT-MP or higher order CT-MP complex such as a heterodimer, e.g., in cases where interspecific binding sequences are present, then the present disclosure provides a plurality of nucleic acid sequences that collectively encode the polypeptides of a CT-MP.

In some cases, the nucleotide sequence is operably linked to one or more transcriptional control elements, e.g., promoters, such as promoters that are functional in a eukaryotic cell, where the promoter can be a constitutive promoter or an inducible promoter.

In some cases, the nucleic acid(s) is/are present in a recombinant expression vector. Thus, this disclosure provides a recombinant expression vector comprising a nucleic acid comprising a nucleotide sequence encoding a CT-MP. In some cases, the present disclosure provides a plurality of recombinant expression vectors that collectively comprise nucleotide sequences encoding two or more polypeptides of a CT-MP such as a heterodimer comprising an interspecific binding sequence.

Suitable expression vectors are well known and include, but are not limited to, viral vectors (e.g., viral vectors based on vaccinia virus; poliovirus; adenovirus; adeno-associated virus; human immunodeficiency virus, and the like). Depending on the host/vector system utilized, any of a number of well-known, suitable transcription and translation control elements, including constitutive and inducible promoters, transcription enhancer elements, transcription terminators, etc. may be used in the expression.

Suitable eukaryotic promoters (promoters functional in a eukaryotic cell) are well known in the art. Selection of the appropriate vector and promoter is well within the level of ordinary skill in the art. The expression vector may also contain a ribosome binding site for translation initiation and a transcription terminator. The expression vector may also include appropriate sequences for amplifying expression.

This disclosure provides a genetically modified host cell, where the host cell is genetically modified with a nucleic acid or a recombinant expression vector encoding a CT-MP. Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines are likewise well known and include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

A CT-MP can be produced using a genetically modified host cell as described herein. Thus, this disclosure provides methods of producing a CT-MP. The methods generally involve culturing, in a culture medium, a host cell (an “expression host cell”) that is genetically modified with a nucleic acid (e.g., a recombinant expression vector) comprising a nucleotide sequence encoding the CT-MP; and isolating the CT-MP from the genetically modified host cell and/or the culture medium.

Isolation of the CT-MP from the expression host cell (e.g., from a lysate of the expression host cell) and/or the culture medium in which the host cell is cultured, can be carried out using standard methods of protein purification.

For example, a lysate may be prepared of the expression host and the lysate purified using high performance liquid chromatography (HPLC), exclusion chromatography, gel electrophoresis, affinity chromatography, or other purification technique. Alternatively, where the CT-MP is secreted from the expression host cell into the culture medium, the CT-MP can be purified from the culture medium using HPLC, exclusion chromatography, gel electrophoresis, affinity chromatography, or other purification technique. In some cases, the compositions which are used will comprise at least 80% by weight, at least about 85% by weight, at least about 95% by weight, or at least about 99.5% by weight, of the CT-MP, in relation to contaminants related to the method of preparation of the product and its purification. The percentages can be based upon total protein.

In some cases, e.g., where the CT-MP comprises an affinity tag, the CT-MP can be purified using an immobilized binding partner of the affinity tag.

### Methods

A CT-MP can be used to increase the number and/or activity of a CAR-T cell. Thus, this disclosure provides methods of increasing the number and/or activity of a CAR-T cell. A CT-MP can be used in a cancer treatment regimen. Thus, this disclosure provides methods of treating cancer in an individual, Methods of increasing the number and/or activity of a CAR-T cell

This disclosure provides methods of increasing the number and/or activity of a CAR-T cell, the methods comprising contacting a CAR-T cell with a CT-MP.

In some cases, a CT-MP, when contacted in vitro or in vivo with a target T cell, e.g., a CAR-T cell, increases proliferation of the target CAR-T cell. For example, in some cases, a CT-MP, when contacted in vitro or in vivo with a target T cell, e.g., a CAR-T cell, increases proliferation of the target T cell by at least 10%, at least 15%, at least 20%, at least 25%, at least 30%, at least 35%, at least 40%, at least 45%, at least 50%, at least 75%, at least 100% (or 2-fold), at least 2.5-fold, at least 5-fold, at least 10-fold, at least 50-fold, at least 100-fold, or more than 100-fold, compared to the proliferation of the target T cell not contacted with the CT-MP or other external agent such as recombinant IL-2. In some cases, following contact with a CT-MP, the number of target T cells increases by at least 10%, at least 15%, at least 20%, at least 25%, at least 30%, at least 35%, at least 40%, at least 45%, at least 50%, at least 75%, at least 100% (or 2-fold), at least 2.5-fold, at least 5-fold, at least 10-fold, at least 50-fold, at least 100-fold, or more than 100-fold, compared to the number of target T cells in the absence of contact with the CT-MP or other external agent such as recombinant IL-2.

In some cases, the administration of one or multiple doses of a pharmaceutical composition comprising a CT-MP, the number of target T cells, e.g., CAR-T cells, increases by at least one order of magnitude, e.g., at least one order of magnitude, at least two orders of magnitude, or at least three orders of magnitude.

In some cases, a CAR-T cell (e.g., a population of CAR-T cells) is contacted in vitro with a CT-MP. For example, in some cases, T cells obtained from a cancer patient (a patient having a cancer) are genetically modified in vitro with a nucleic acid comprising a nucleotide sequence encoding a CAR comprising an antigen-binding portion (e.g., an antibody (e.g., a scFv or a nanobody)) that is specific for a cancer-associated antigen present on cancer cells in the patient, to generate CAR-T cells. In some cases, the CAR-T cells are contacted in vitro with a CT-MP that comprises a CAR-BP that binds specifically to the CAR, to generate an in vitro population of expanded and/or activated CAR-T cells. The in vitro population of expanded and/or activated CAR-T cells can then be administered to the patient to treat the cancer.

In some cases, a CAR-T cell (e.g., a population of CAR-T cells) is contacted in vivo with a CT-MP. Thus, in some cases, a method of this disclosure comprises administering to an individual (e.g., a cancer patient) an effective amount of a CT-MP, or a pharmaceutical composition comprising the CT-MP, where the individual has been treated with CAR-T cells that express a target CAR. A target CAR is one that comprises an antigen-binding portion (e.g., an antibody (e.g., a scFv or a nanobody)) that is specific for a cancer-associated antigen present on cancer cells in the patient, where the CAR is recognized and bound by the CAR-BP (e.g., antibody or CAA) present in the CT-MP. For example, where the individual has a B-cell lymphoma, the target CAR may include a scFv specific for CD19, and the CT-MP can include, as the CAR-BP, a CD19 polypeptide. As another example, where the individual has a B-cell lymphoma, the target CAR may include a scFv specific for CD20, and the CT-MP can include, as the CAR-BP, a CD20 polypeptide.

Combination Therapy with CAR-T Cells

A CT-MP can be co-administered with CAR-T cells. Thus, this disclosure provides a combination therapy method for treating cancer. In some cases, the method comprises: a) administering to the individual a population of CAR-T cells that express on their surface a target CAR; and b) administering to the individual an effective amount of a pharmaceutical composition according to claim 18, wherein the CAR-BP of the CT-MP specifically binds to the target CAR.

The administration of the CT-MP and the CAR-T cells can be substantially simultaneous, e.g., the CT-MP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) before or after of administration of the at CAR-T cells. Alternatively, the CT-MP can be administered to an individual from 1 day to about 2 weeks (e.g., from 1 day to 2 days, from 2 days to 7 days, or from 1 week to 2 weeks) after administration of the CAR-T cells. Thereafter, the CT-MP may be re-administered periodically, as determined by the patient's physician, to maintain the desired level of CAR-T cells in the patient. For example, the CT-MP may be administered every week, every two weeks, every three weeks, monthly, or less frequently than monthly. The CT-MP also may be administered more frequently following the initial administration of CAR-T cells, and then less frequently once the desired level of CAR-T cells has been reached. The level of CAR-T cells in the patient can be readily determined, e.g., using fluorescence-activated cell sorting (FACS), e.g., using a fluorescently labeled antibody that binds specifically to the CAR-T cells.

In some cases, the number of CAR-T cells that are administered in a combination therapy is less than the number of CAR-T cells that are administered in CAR-T monotherapy. For example, according to the FDA approved package insert, where CAR-T cells are genetically modified to produce Yescarta®, the number of such CAR-T cells that are administered in a monotherapy regimen to an individual having a CD19⁺ cancer (e.g., a B cell lymphoma such as large B-cell lymphoma of follicular lymphoma) is about 2×10⁶ CAR-positive viable T cells per kg body weight up to a maximum of 2×10⁸ CAR-positive viable T cells. In a combination therapy with a CT-MP, however, a lesser number of Yescarta CAR-T cells may be administered to the patient.

A dose of CAR-T cells that is co-administered in a combination therapy of this disclosure can be one, two, three or more orders of magnitude less than the number of CAR-T cells that are administered in a monotherapy setting without co-administration of a CT-MP. For example, in the case of Yescarta, the number of such CAR-T cells that are administered in a monotherapy regimen to an individual having a CD19⁺ cancer (e.g., a B cell lymphoma such as large B-cell lymphoma of follicular lymphoma) is about 2×10⁵ CAR-positive viable T cells per kg body weight, about 2×10⁴ CAR-positive viable T cells per kg body weight, or about 2×10³ CAR-positive viable T cells per kg body weight, in each case preceded and/or followed by administration of the CT-MP to increase the number of Yescarta T cells in the patient. Alternatively, a dose of CAR-T cells that is co-administered in a combination therapy of this disclosure can be from 10% to about 90% or more lower (e.g., from 10% to 20%, from 20% to 30%, from 30% to 40%, from 40% to 50%, from 50% to 60%, from 60% to 70%, from 70% to 80%, from 80% to 90%, or greater than 90%) than the number of CAR-T cells that are administered in a monotherapy setting without co-administration of a CT-MP.

In some cases, a method of this disclosure for treating a cancer does not comprise a lymphodepleting regimen. In other words, in some cases, an individual being treated with a CT-MP has not undergone lymphodepleting chemotherapy as part of a CAR-T cell therapy. Lymphodepleting chemotherapy can comprise multiple administrations of, e.g., cyclophosphamide and fludarabine before infusion of CAR-T cells (e.g., from 2 days to 7 days before infusion of CAR-T cells).

In some cases, a method of this disclosure for treating a cancer does not comprise treatment with recombinantly produced IL-2, e.g., aldesleukin (Proleukin®) following administration of CAR-T cells.

In some cases, in some cases, an individual being treated with a CT-MP does not undergo lymphodepleting chemotherapy as part of a CAR-T cell therapy, and also does not receive recombinantly produced IL-2, e.g., aldesleukin (Proleukin®) following administration of CAR-T cells.

In some cases, a CT-MP comprises a CD19 polypeptide, a CD20 polypeptide, or a BCMA polypeptide and is used to treat a hematological cancer. Hematological cancers include, e.g., B-cell non-Hodgkin lymphoma, pediatric B-cell acute lymphoblastic leukemia, multiple myeloma, chronic lymphocytic leukemia, acute myeloid leukemia, T-cell lymphomas, and Hodgkin lymphoma.

Combination Therapy with Additional Therapeutic Agents

In some cases, a method of this disclosure for treating cancer in an individual comprises: a) administering a CT-MP to an individual who has received CAR-T cells; and b) administering at least one additional therapeutic agent or therapeutic treatment. Suitable additional therapeutic agents include, but are not limited to, small molecule cancer chemotherapeutic agents and immune checkpoint inhibitors. Suitable additional therapeutic treatments include, e.g., radiation, surgery (e.g., surgical resection of a tumor), and the like.

A treatment method of this disclosure can comprise co-administration of a CT-MP and at least one additional therapeutic agent. By “co-administration” is meant that both a CT-MP and at least one additional therapeutic agent are administered to an individual, although not necessarily at the same time, in order to achieve a therapeutic effect that is the result of having administered both the CT-MP and the at least one additional therapeutic agent. The administration of the CT-MP and the at least one additional therapeutic agent can be substantially simultaneous, e.g., the CT-MP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the at least one additional therapeutic agent. In some cases, a CT-MP is administered to an individual who is undergoing treatment with, or who has undergone treatment with, the at least one additional therapeutic agent. The administration of the CT-MP can occur at different times and/or at different frequencies.

As an example, a treatment method of this disclosure can comprise co-administration of a CT-MP and at least one immune checkpoint inhibitor such as an antibody specific for an immune checkpoint. By “co-administration” is meant that both a CT-MP and an immune checkpoint inhibitor (e.g., an antibody specific for an immune checkpoint polypeptide) are administered to an individual, although not necessarily at the same time, in order to achieve a therapeutic effect that is the result of having administered both the CT-MP and the immune checkpoint inhibitor (e.g., an antibody specific for an immune checkpoint polypeptide). The administration of the CT-MP and the immune checkpoint inhibitor (e.g., an antibody specific for an immune checkpoint polypeptide) can be substantially simultaneous, e.g., the CT-MP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, within about 24 hours, or sequentially, i.e., within 1 week, 3 weeks 3 weeks, four weeks or a month following administration of the immune checkpoint inhibitor (e.g., an antibody specific for an immune checkpoint polypeptide). In some cases, a CT-MP is administered to an individual who is undergoing treatment with, or who has undergone treatment with, an immune checkpoint inhibitor (e.g., an antibody specific for an immune checkpoint polypeptide). The administration of the CT-MP and the immune checkpoint inhibitor (e.g., an antibody specific for an immune checkpoint polypeptide) can occur at different times and/or at different frequencies. Where there is an established dosing interval for the checkpoint inhibitor, depending on the interval, it may be possible to administer the CT-MP on the same day as the checkpoint inhibitor. For example, in some cases, where the dosing schedule for pembrolizumab is once every three weeks, the pharmaceutical composition comprising the CT-MP may be administered on the same day.

Exemplary immune checkpoint inhibitors include inhibitors that target an immune checkpoint polypeptide such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122 and CD137. In some cases, the immune checkpoint polypeptide is an inhibitory checkpoint molecule that targets A2AR, B7-H3, B7-H4, BTLA, CTLA-4, IDO, KIR, LAG3, PD-1, TIM3, CD96, TIGIT and VISTA. In some cases, the immune checkpoint polypeptide is an inhibitory checkpoint molecule that targets CTLA-4, LAG3, PD-1 or TIGIT, e.g., an antibody that targets CTLA-4, LAG3, PD-1, PD-L1, or TIGIT.

In some cases, the immune checkpoint inhibitor is an antibody (as that term is described herein) specific for an immune checkpoint polypeptide. In some cases, the anti-immune checkpoint antibody is a monoclonal antibody. In some cases, the anti-immune checkpoint antibody is humanized, or de-immunized such that the antibody does not substantially elicit an immune response in a human. In some cases, the anti-immune checkpoint antibody is a humanized monoclonal antibody. In some cases, the anti-immune checkpoint antibody is a de-immunized monoclonal antibody. In some cases, the anti-immune checkpoint antibody is a fully human monoclonal antibody. In some cases, the anti-immune checkpoint antibody inhibits binding of the immune checkpoint polypeptide to a ligand for the immune checkpoint polypeptide. In some cases, the anti-immune checkpoint antibody inhibits binding of the immune checkpoint polypeptide to a receptor for the immune checkpoint polypeptide.

Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (Medlmmune/AZ), CP-870,893 (Genentech), Mogamulizumab (Kyowa Hakko Kirin), Varlilumab (CelIDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte); KN035; and combinations thereof. For example, in some cases, the immune checkpoint inhibitor is an anti-PD-1 antibody. Suitable anti-PD-1 antibodies include, e.g., nivolumab, pembrolizumab (also known as MK-3475), pidilizumab, SHR-1210, PDR001, and AMP-224. In some cases, the anti-PD-1 monoclonal antibody is nivolumab, pembrolizumab or PDR001. Suitable anti-PD1 antibodies are described in U.S. Patent Publication No. 2017/0044259. For pidilizumab, see, e.g., Rosenblatt et al. (2011) J. Immunother. 34:409-18. In some cases, the immune checkpoint inhibitor is an anti-CTLA-4 antibody. In some cases, the anti-CTLA-4 antibody is ipilimumab or tremelimumab. For tremelimumab, see, e.g., Ribas et al. (2013) J. Clin. Oncol. 31:616-22. In some cases, the immune checkpoint inhibitor is an anti-PD-L1 antibody. In some cases, the anti-PD-L1 monoclonal antibody is BMS-935559, MED14736, MPDL3280A (also known as RG7446), KN035, or MSB0010718C. In some cases, the anti-PD-L1 monoclonal antibody is MPDL3280A (atezolizumab) or MED14736 (durvalumab). For durvalumab, see, e.g., WO 2011/066389. For atezolizumab, see, e.g., U.S. Pat. No. 8,217,149. In some cases, the immune checkpoint inhibitor is an anti-TIGIT antibody that binds to T-cell immunoreceptor with immunoglobulin and ITIM domains (TIGIT). In some cases, the anti-TIGIT antibody is BMS-986207 (Bristol-Myers Squibb). In some cases, the anti-TIGIT antibody is tiragolumab. In some cases, the anti-TIGIT antibody is EOS88448 (EOS-448). See, e.g., U.S. Pat. Nos. 11,008,390 and 10,189,902; U.S. Patent Publication No. 2017/0088613; and WO 2019/137541. In some cases, the anti-LAG3 antibody is Relatlimab (Bristol-Myers Squibb).

Among such checkpoint inhibitors, antibodies to PD-1, PD-L1, CTLA-4, TIGIT and LAG3 are the most common, with at least nivolumab, tremelimumab, pembrolizumab, ipilimumab, cemiplimab, atezolizumab, avelumab, tisleizumab, durvalumab and relatlimab having been approved by the FDA and/or regulatory agencies outside of the U.S. A CT-MP of this disclosure also may be co-administered with combinations of checkpoint inhibitors, e.g., a combination of (i) an antibody to PD-1 or PD-L1, (ii) an antibody to CTLA-4, (iii) an antibody to TIGIT and/or (iv) an antibody to LAG3.

### Subjects Suitable for Treatment

Subjects suitable for treatment with a method of this disclosure include individuals who have cancer, including individuals who have been newly diagnosed as having cancer, individuals who have been treated for cancer but who failed to respond to the treatment, and individuals who have been treated for cancer and who initially responded but subsequently relapse and/or became refractory to the treatment. Subjects suitable for treatment include individuals having a cancer in which the cancer cells express, or overexpress, a cancer-associated antigen.

In some cases, the subject is an individual who is undergoing treatment with an immune checkpoint inhibitor. In some cases, the subject is an individual who has undergone treatment with an immune checkpoint inhibitor, but whose disease has progressed despite having received such treatment. In some cases, the subject is an individual who is undergoing treatment with, or who has undergone treatment with, a cancer chemotherapeutic agent. In some cases, the subject is an individual who is preparing to undergo treatment with, is undergoing treatment with, or who has undergone treatment with, an immune checkpoint inhibitor. In some cases, the subject is an individual who is preparing to undergo treatment with, is undergoing treatment with, or who has undergone treatment with, a cancer chemotherapeutic agent, radiation treatment, surgery, and/or treatment with another therapeutic agent.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A cell therapy modulatory polypeptide (CT-MP) that modulates a T cell that has been modified to display an antigen-binding polypeptide such as a chimeric antigen receptor (CAR), comprising:
a) one or more T cell modulatory polypeptides;
b) a polypeptide that specifically binds to an antigen binding portion of a target T cell, e.g., a polypeptide (a CAR-BP) that specifically binds to an antigen binding portion of a target CAR on a CAR-T cell; and
c) a scaffold component that can display both a polypeptide that specifically binds to an antigen binding portion of a target T cell, e.g., a CAR-BP, and a T cell modulatory polypeptide (TMP),
wherein the scaffold component is, for example, a lipid vesicle (e.g., liposome) or micelle, an artificial antigen presenting cell such as an engineered erythroid cell or enucleated cell (e.g., platelets), or an immunoglobulin (Ig) Fc polypeptide,
wherein the CT-MP may comprise one or more independently selected linkers between the components,
optionally wherein the T cell is a CAR-T cell, and wherein the CAR-T cell is in a pharmaceutical composition of Yescarta®, Tecartus®, Kymriah®, Abecma® or Breyanzi®.

Aspect 2. A CT-MP of Aspect 1, wherein the CT-MP comprises, in order from N-terminus to C-terminus:
a1) one or more T cell modulatory polypeptides;
b1) an optional linker;
c1) the CAR-BP;
d1) an optional linker; and
e1) the Ig Fc polypeptide; or
a2) one or more T cell modulatory polypeptides;
b2) an optional linker;
c2) the Ig Fc polypeptide; and
d2) an optional linker;
e2) the CAR-BP;
a3) one or more T cell modulatory polypeptides;
b3) the CAR-BP
c3) an optional linker;
d3) the Ig Fc polypeptide; and
e3) one or more T cell modulatory polypeptides;
a4) the CAR-BP;
b4) an optional linker;
c4) one or more T cell modulatory polypeptides; and
b4) an optional linker;
d4) the Ig Fc polypeptide.
a5) the CAR-BP;
b5) an optional linker;
c5) the Ig Fc polypeptide.
d5) an optional linker;
e5) one or more T cell modulatory polypeptides.

Aspect 3. A CT-MP of any Aspect 1 or 2, wherein at least one of the one or more one or more T cell modulatory polypeptides: (i) causes activation and/or proliferation of the CAR-T cell, optionally a cytokine such as a wild-type or variant of IL-2, a 4-1BBL polypeptide, an ICOS-L polypeptide, an OX-40L polypeptide, a CD80 polypeptide, a CD86 polypeptide, a CD40 polypeptide, a CD70 polypeptide, or a combination thereof, or (ii) causes suppression/inhibition of the CAR-T cell, optionally a PD-L1, PD-L2 or FasL polypeptide or a combination thereof, or (iii) binds to and inhibits a checkpoint on a T cell, optionally wherein the checkpoint inhibitor is an antibody or binding fragment thereof that binds to PD-1, CTLA-4, TIGIT or LAG3.

Aspect 4. A CT-MP of any one of Aspects 1-3, wherein the one or more T cell modulatory polypeptides comprises an IL-2 polypeptide, optionally wherein the IL-2 polypeptide is a variant IL-2 polypeptide.

Aspect 5. A CT-MP of Aspect 3, wherein the one or more T cell-modulatory polypeptide comprises two IL-2 polypeptides, optionally wherein the two IL-2 polypeptides are each variant IL-2 polypeptides.

Aspect 6. A CT-MP of any one of Aspects 4 or 5, wherein the CT-MP comprises, in order from N-terminus to C-terminus:
a1) a first IL-2 polypeptide;
b1) an optional linker;
c1) a second IL-2 polypeptide;
d1) an optional linker;
e1) the CAR-BP;
f1) an optional linker; and
g1) the Ig Fc polypeptide; or
a2) the CAR-BP;
b2) an optional linker;
c2) the Ig Fc polypeptide;
d2) a first IL-2 polypeptide;
e2) an optional linker; and
f2) a second IL-2 polypeptide; or
a3) the CAR-BP;
b3) an optional linker;
c3) a first IL-2 polypeptide;
d3) an optional linker;
e3) a second IL-2 polypeptide; and
f3) the Ig Fc polypeptide.

Aspect 7. A CT-MP of any of Aspects 4-6, wherein at least one of the one or more IL-2 polypeptides is a variant IL-2 polypeptide that binds to an IL-2 receptor (IL-2R) having α, β, and γ chains comprising amino acid sequences depicted in FIG. 16B-16D and exhibits reduced affinity compared to the affinity of a wild-type IL-2 polypeptide for the same IL-2R.

Aspect 8. A CT-MP of Aspect 7, wherein at least one of the one or more variant IL-2 polypeptides binds to the IL-2R alpha chain and the IL-2R beta chain with an affinity that is less than the affinity of a wild-type IL-2 polypeptide for the IL-2R alpha chain and the IL-2R beta chain when assayed under the same conditions in a BLI assay, optionally wherein the binding affinity for human IL-2Rα and IL-2Rβ is decreased by at least about 100 fold and at least about 3-fold, respectively, compared with the binding of wild-type IL-2 binding for the IL-2R alpha chain and the IL-2R beta chain.

Aspect 9. A CT-MP of Aspect 7 or 8, wherein at least one of the one or more variant IL-2 polypeptides comprises an amino acid sequence having at least 95% amino acid sequence identity to the amino acid sequence depicted in FIG. 16A.

Aspect 10. A CT-MP of Aspect 9, wherein the variant IL-2 polypeptide comprises: i) an H16A substitution and an F42A substitution; or ii) an H16T substitution and an F42A substitution.

Aspect 11. A CT-MP of any one of Aspects 1-10, wherein the scaffold component is an Ig Fc that substantially does not induce cell lysis.

Aspect 12. A CT-MP of Aspect 11, wherein the Ig Fc polypeptide is an IgG1 Fc polypeptide that comprises one or more amino acid substitutions selected from N297A, L234A, L235A, L234F, L235E, and P331S, wherein N297, L234, L23A, and P331 correspond to N77, L14, L15, and P111, respectively, of the amino acid sequences depicted in FIG. 15A.

Aspect 13. A CT-MP of any one of Aspects 1-12, wherein the Ig Fc polypeptide comprises an amino acid sequence having at least 95% amino acid sequence identity to the amino acid sequence depicted in any one of FIG. 15B-15I.

Aspect 14. A CT-MP of any one of Aspects 1-13, wherein the CAR-BP comprises an antigen binding portion or an antibody or non-antibody protein.

Aspect 15. A CT-MP of any one of Aspects 1-13, wherein the CAR-BP comprises a cancer-associated antigen.

Aspect 16. A CT-MP of Aspect 15, wherein the CAR-BP comprises:
a) a cancer-associated antigen selected from a solid tumor-associated antigen selected from: EGFR, HER2, EGFR806, mesothelin, PSCA, MUC1, claudin 18.2, EpCAM, GD2, VEGFR2, AFP, Nectin4/FAP, CEA, LewisY, Glypican-3, EGFRIII, IL-13Rα2, CD171, MUC16, PSMA, AXL, CD20, CD80/86, c-MET, DLL-3, DR5, EpHA2, FR-α, gp100, MAGE-A1, MAGE-A3, MAGE-A4, and LMP1; or
b) a cancer-associated antigen associated with hematological cancer, wherein the cancer-associated antigen is selected from: BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, IGF-R1, IL6, KIR, PD-1, PD-L1, TRAILR1, TRAILR2, transferrin receptor, and VEGF.

Aspect 17. A CT-MP of any one of Aspects 1-16, wherein the target CAR binds to:
a) a cancer-associated antigen selected from a solid tumor-associated antigen selected from: EGFR, HER2, EGFR806, mesothelin, PSCA, MUC1, claudin 18.2, EpCAM, GD2, VEGFR2, AFP, Nectin4/FAP, CEA, LewisY, Glypican-3, EGFRIII, IL-13Rα2, CD171, MUC16, PSMA, AXL, CD20, CD80/86, c-MET, DLL-3, DR5, EpHA2, FR-α, gp100, MAGE-A1, MAGE-A3, MAGE-A4, and LMP1; or
b) a cancer-associated antigen associated with hematological cancer, wherein the cancer-associated antigen is selected from: BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, IGF-R1, IL6, KIR, PD-1, PD-L1, TRAILR1, TRAILR2, transferrin receptor, and VEGF.

Aspect 18. A CT-MP comprising a dimer of two CT-MPs according to any one of Aspects 1-17, wherein the first and second CT-MPs are covalently bound by one or more disulfide bonds between the Ig Fc polypeptides of the two CT-MPs, optionally wherein the Ig Fc polypeptides of the two CT-MPs do not comprise complementary interspecific binding sequences, or optionally wherein the Ig Fc polypeptides of the two CT-MPs do comprise complementary interspecific binding sequences.

Aspect 19. A pharmaceutical composition comprising a CT-MP of any one of Aspects 1-18.

Aspect 20. A composition comprising one or more nucleic acids encoding a CT-MP of any one of Aspects 1-18.

Aspect 21. A composition comprising one or more expression vectors, wherein the one or more expression vectors comprise one or more nucleotide sequences encoding a CT-MP of any one of Aspects 1-18.

Aspect 22. A host cell genetically modified with the composition of one or more nucleic acids of Aspect 20 or composition of one or more recombinant expression vectors of Aspect 21.

Aspect 23. A method of producing a CT-MP, the method comprising culturing a genetically modified host cell according to Aspect 21 under conditions such that the genetically modified host cell produces the CT-MP.

Aspect 24. A method of increasing the number and/or activity of a CAR-T cell in an individual, the method comprising administering to the individual an effective amount of a CT-MP of any one of Aspects 1-18, or the pharmaceutical composition of Aspect 19, wherein the individual has been treated with CAR-T cells that express the target CAR, optionally wherein the individual has been treated with Yescarta® (axicabtagene ciloleucel), Tecartus® (brexucabtagene autoleucel), Kymriah® (tisagenleucleucel), Abecma® (idecabtagene vicleucel), or Breyanzi® (lisocabtagene maraleucel).

Aspect 25. A method of treating a cancer in an individual, the method comprising: a) administering to the individual a population of CAR-T cells that express on their surface a target CAR; and b) administering to the individual an effective amount of a pharmaceutical composition according to Aspect 19, wherein the CAR-BP of the CT-MP specifically binds to the target CAR.

Aspect 26. The method of Aspect 25, wherein the individual did not undergo lymphodepleting chemotherapy as part of a CAR-T cell therapy.

Aspect 27. The method of Aspect 25 or Aspect 26, wherein, following administration of the CAR-T cells, the individual does not receive a pharmaceutical composition comprising IL-2 polypeptide an IL-2 polypeptide other than the CT-MP as part of the CAR-T cell therapy.

Aspect 28. The method of any one of Aspects 25-27, comprising co-administering at least one immune checkpoint inhibitor to the patient, optionally wherein the at least one immune checkpoint inhibitor comprises an antibody specific for PD-L1, PD-1, TIGIT, LAG3 or CTLA4.

While this disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of this disclosure. All such modifications are intended to be within the scope of the claims appended hereto.

What is claimed is:

1. A cell therapy modulatory polypeptide (CT-MP) that modulates a target T cell, wherein the target T cell is a CAR-T cell that comprises a chimeric antigen receptor (CAR), and wherein the CT-MP comprises:
a) one or more T cell modulatory polypeptides;
b) a polypeptide that specifically binds to an antigen binding portion of a target CAR (a CAR-BP); and
c) a scaffold component, optionally wherein the scaffold component is an immunoglobulin (Ig) Fc polypeptide,
wherein the CT-MP may comprise one or more independently selected linkers between the components,
optionally wherein the CAR-T cell is a Yescarta® T cell, a Tecartus® T cell, a Kymriah® T cell, an Abecma® T cell, or a Breyanzi® T cell.

2. A CT-MP of claim 1, wherein the CT-MP comprises, in order from N-terminus to C-terminus:
a1) one or more T cell modulatory polypeptides;
b1) an optional linker;
c1) the CAR-BP;
d1) an optional linker; and
e1) the Ig Fc polypeptide; or
a2) one or more T cell modulatory polypeptides;
b2) an optional linker;
c2) the Ig Fc polypeptide; and
d2) an optional linker;
e2) the CAR-BP;
a3) one or more T cell modulatory polypeptides;
b3) the CAR-BP
c3) an optional linker;
d3) the Ig Fc polypeptide; and
e3) one or more T cell modulatory polypeptides;
a4) the CAR-BP;
b4) an optional linker;
c4) one or more T cell modulatory polypeptides; and
b4) an optional linker;
d4) the Ig Fc polypeptide.
a5) the CAR-BP;
b5) an optional linker;
c5) the Ig Fc polypeptide.
d5) an optional linker;
e5) one or more T cell modulatory polypeptides.

3. A CT-MP of any claim 1 or 2, wherein at least one of the one or more one or more T cell modulatory polypeptides is a polypeptide that
(i) causes activation and/or proliferation of the CAR-T cell, optionally a cytokine such as a wild-type or variant of IL-2, a 4-1BBL polypeptide, an ICOS-L polypeptide, an OX-40L polypeptide, a CD80 polypeptide, a CD86 polypeptide, a CD40 polypeptide, a CD70 polypeptide, and combinations thereof, or
(ii) causes suppression/inhibition of the CAR-T cell, optionally a PD-L1, PD-L2 or FasL polypeptide or a combination thereof, or
(iii) binds to and inhibits an immune checkpoint on the CAR-T cell, optionally wherein the checkpoint inhibitor is an antibody or binding fragment thereof that binds to PD-1, CTLA-4, TIGIT or LAG3.

4. A CT-MP of any one of claims 1-3, wherein the one or more T cell modulatory polypeptides comprises an IL-2 polypeptide, optionally wherein the IL-2 polypeptide is a variant IL-2 polypeptide.

5. A CT-MP of claim 3, wherein the one or more T cell-modulatory polypeptide comprises two IL-2 polypeptides optionally wherein the two IL-2 polypeptides are variant IL-2 polypeptides.

6. A CT-MP of any one of claims 4 or 5, wherein the CT-MP comprises, in order from N-terminus to C-terminus:
a1) a first IL-2 polypeptide;
b1) an optional linker;
c1) a second IL-2 polypeptide;
d1) an optional linker;
e1) the CAR-BP;
f1) an optional linker; and
g1) the Ig Fc polypeptide; or
a2) the CAR-BP;
b2) an optional linker;
c2) the Ig Fc polypeptide;
d2) a first IL-2 polypeptide;
e2) an optional linker; and
f2) a second IL-2 polypeptide; or
a3) the CAR-BP;
b3) an optional linker;
c3) a first IL-2 polypeptide;
d3) an optional linker;
e3) a second IL-2 polypeptide; and
f3) the Ig Fc polypeptide.

7. A CT-MP of any of claims 4-6, wherein at least one of the one or more IL-2 polypeptides is a variant IL-2 polypeptide that binds to an IL-2 receptor (IL-2R) having α, β, and γ chains comprising amino acid sequences depicted in FIG. 16B-16D and exhibits reduced affinity compared to the affinity of a wild-type IL-2 polypeptide for the same IL-2R.

8. A CT-MP of claim 7, wherein at least one of the one or more variant IL-2 polypeptides binds to the IL-2R alpha chain and the IL-2R beta chain with an affinity that is less than the affinity of a wild-type IL-2 polypeptide for the IL-2R alpha chain and the IL-2R beta chain when assayed under the same conditions in a BLI assay, optionally wherein the binding affinity for human IL-2Rα and IL-2Rβ is decreased by at least about 100 fold and at least about 3-fold, respectively, compared with the binding of wild-type IL-2 binding for the IL-2R alpha chain and the IL-2R beta chain.

9. A CT-MP of claim 7 or 8, wherein at least one of the one or more variant IL-2 polypeptides comprises an amino acid sequence having at least 95% amino acid sequence identity to the amino acid sequence depicted in FIG. 16A.

10. A CT-MP of claim 9, wherein the variant IL-2 polypeptide comprises: i) an H16A substitution and an F42A substitution; or ii) an H16T substitution and an F42A substitution.

11. A CT-MP of any one of claims 1-10, wherein the scaffold component is an Ig Fc polypeptide that substantially does not induce cell lysis.

12. A CT-MP of claim 11, wherein the Ig Fc polypeptide comprises an IgG1 Fc polypeptide having one or more amino acid substitutions selected from N297A, L234A, L235A, L234F, L235E, and P331S, wherein N297, L234, L23A, and P331 correspond to N77, L14, L15, and P111, respectively, of the amino acid sequences depicted in FIG. 15A.

13. A CT-MP of any one of claims 1-12, wherein the Ig Fc polypeptide comprises an amino acid sequence having at least 95% amino acid sequence identity to the amino acid sequence depicted in any one of FIG. 15B-15I.

14. A CT-MP of any one of claims 1-12, wherein the CAR-BP comprises:
a) an antigen binding portion or an antibody or non-antibody protein; or
b) a cancer-associated antigen.

15. A CT-MP of claim 14, wherein the CAR-BP comprises:
a) a cancer-associated antigen selected from a solid tumor-associated antigen selected from:
EGFR, HER2, EGFR806, mesothelin, PSCA, MUC1, claudin 18.2, EpCAM, GD2, VEGFR2, AFP, Nectin4/FAP, CEA, LewisY, Glypican-3, EGFRIII, IL-13Rα2, CD171, MUC16, PSMA, AXL, CD20, CD80/86, c-MET, DLL-3, DR5, EpHA2, FR-α, gp100, MAGE-A1, MAGE-A3, MAGE-A4, and LMP1; or
b) a cancer-associated antigen associated with hematological cancer, wherein the cancer-associated antigen is selected from: BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, IGF-R1, IL6, KIR, PD-1, PD-L1, TRAILR1, TRAILR2, transferrin receptor, and VEGF.

16. A CT-MP of any one of claims 1-15, wherein the target CAR binds to:
a) a cancer-associated antigen selected from a solid tumor-associated antigen selected from: EGFR, HER2, EGFR806, mesothelin, PSCA, MUC1, claudin 18.2, EpCAM, GD2, VEGFR2, AFP, Nectin4/FAP, CEA, LewisY, Glypican-3, EGFRIII, IL-13Rα2, CD171, MUC16, PSMA, AXL, CD20, CD80/86, c-MET, DLL-3, DR5, EpHA2, FR-α, gp100, MAGE-A1, MAGE-A3, MAGE-A4, and LMP1; or
b) a cancer-associated antigen associated with hematological cancer, wherein the cancer-associated antigen is selected from: BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, IGF-R1, IL6, KIR, PD-1, PD-L1, TRAILR1, TRAILR2, transferrin receptor, and VEGF.

17. A CT-MP comprising a dimer of two CT-MPs according to any one of claims 1-16, wherein the first and second CT-MPs are covalently bound by one or more disulfide bonds between the Ig Fc polypeptides of the two CT-MPs.

18. A pharmaceutical composition comprising a CT-MP of any one of claims 1-17.

19. A composition comprising one or more nucleic acids encoding a CT-MP of any one of claims 1-17.

20. A composition comprising one or more expression vectors, wherein the one or more expression vectors comprise one or more nucleotide sequences encoding a CT-MP of any one of claims 1-17.

21. A host cell genetically modified with the composition of one or more nucleic acids of claim 19 or composition of one or more recombinant expression vectors of claim 20.

22. A method of producing a CT-MP, the method comprising culturing a genetically modified host cell according to claim 21 under conditions such that the genetically modified host cell produces the CT-MP.

23. A method of increasing the number and/or activity of a CAR-T cell in an individual, the method comprising administering to the individual an effective amount of a CT-MP of any one of claims 1-17, or the pharmaceutical composition of claim 18, wherein the individual has been treated with T cells that express the target CAR, optionally wherein the individual has been treated with Yescarta® (axicabtagene ciloleucel), Tecartus® (brexucabtagene autoleucel), Kymriah® (tisagenleucleucel), Abecma® (idecabtagene vicleucel), or Breyanzi® (lisocabtagene maraleucel).

24. A method of treating a cancer in an individual, the method comprising:
a) administering to the individual a population of CAR-T cells that express on their surface a target CAR; and
b) administering to the individual an effective amount of a pharmaceutical composition according to claim 18, wherein the CAR-BP of the CT-MP specifically binds to the target CAR.

25. The method of claim 24, wherein the individual did not undergo lymphodepleting chemotherapy as part of a CAR-T cell therapy.

26. The method of claim 24 or 25, wherein, following administration of the CAR-T cells, the individual does not receive a pharmaceutical composition comprising IL-2 polypeptide other than the CT-MP as part of the CAR-T cell therapy.

27. The method of any one of claims 24-26, comprising co-administering at least one immune checkpoint inhibitor to the patient, optionally wherein the at least one immune checkpoint inhibitor comprises an antibody specific for PD-L1, PD-1, TIGIT, LAG3 or CTLA4.
